(19) United States
(12) Patent Application Publication
(10) Pub. No.: US 2004/0162477 A1
(43) Pub. Date: Aug. 19, 2004

(54) Apparatus for detecting magnetic fluid identifying sentinel-lymph node

(75) Inventors: Toshiro Okamura, Tokyo (JP); Hitoshi Ueno, Tokyo (JP); Mamoru Kaneko, Hanno-shi (JP); Tomoaki Ueda, Kyoto-shi (JP)

Correspondence Address: SCULLY SCOTT MURPHY & PRESSER, PC, 400 GARDEN CITY PLAZA, GARDEN CITY, NY, 11530

(73) Assignee: OLYMPUS CORPORATION, TOKYO (JP)

(21) Appl. No.: 10/678,504

(22) Filed: Oct. 3, 2003

(30) Foreign Application Priority Data
Oct. 4, 2002 (JP) 2002-292694
Apr. 10, 2003 (JP) 2003-106862

(51) Int. Cl. A61B 5/05

(52) U.S. Cl. 600/409

(57) ABSTRACT

A magnetic fluid detecting apparatus for identifying sentinel lymph nodes comprises a probe, which can be inserted into the body cavity, including an exciting magnet and magnetic sensors within the tip side thereof, and a control device, connected to the probe through a connecting cable, for controlling the probe. With the probe, the exciting magnet and the magnetic sensors are vibrated in the longitudinal direction by an actuator so as to modulate the local magnetic field due to magnetic fluid excited by the exciting magnet, the distortion of the local magnetic field distribution (the change in the magnetic flux density) is detected by the two magnetic sensors, the difference between the outputs from the two magnetic sensors is obtained and subjected to demodulation, and accordingly, the magnetic noise other than the modulation frequency from the terrestrial magnetism or other electric devices is removed, thereby detecting magnetic fluid and identifying sentinel lymph nodes.

This application claims benefit of Japanese Patent Application Nos. 2002-292694 filed in Japan on Oct. 4, 2002, and 2003-106862 filed in Japan on Apr. 10, 2003, the contents of which are incorporated by this reference.

### BACKGROUND OF THE INVENTION

1. Field of the Invention

The present invention relates to a magnetic fluid detecting apparatus for identifying sentinel lymph nodes wherein distribution of the magnetic fluid injected around tumors after a predetermined time period is measured, and the sentinel lymph nodes, where the tumor cells which spread from the origin of the tumor to lymphatic vessels reach in the first stage, are identified thereupon.

2. Related Art Statement

In recent years, excision surgery is frequently performed for cancer in the early stage due to advanced diagnosis of cancer in the early stages. In general, in many cases, surgery for cancer in the early stage is performed for a complete recovery, and accordingly, excision is performed not only for the affected portion, but also for multiple lymph nodes, where metastasis of cancer could occur, situated around the affected portion. Furthermore, with the surgery for cancer in the early stage, following surgery, pathological examination is performed for the excised lymph nodes for confirming whether or not the cancer has spread to lymph nodes, and the treatment method or the like following the surgery is determined based thereupon.

In the surgery stage, whether or not the cancer has spread to lymph nodes is unknown. Accordingly, excision is performed for multiple lymph nodes near the affected portion in the surgery for cancer in the early stage, leading to the great burden on the patient. On the other hand, the probability of metastasis of cancer to lymph nodes is around 20% with regard to breast cancer in the early stage. Accordingly, excessive excision of lymph nodes has been performed for 80% of the patients without actual metastasis of cancer.

In recent years, both of the QOL (Quality of Life) of patients and complete recovery are required in excision surgery for cancer. As a method for the above-described purpose, sentinel node navigation surgery for preventing excessive excision of lymph nodes wherein metastasis of cancer has not occurred is attracting attention. Description will be made below in brief regarding the sentinel navigation surgery.

Recent studies have revealed that in the event that metastasis of cancer to lymph nodes occurs, the metastasis does not occur at random, but occurs following a regular pattern, from the affected portion to the lymph nodes through lymph vessels. It is believed that in the event that metastasis of cancer to the lymph nodes has occurred, metastasis of cancer to sentinel lymph nodes has always occurred. Here, the sentinel lymph node is a lymph node where the cancer cells, which spread from the affected portion to the lymph vessels, reach in the first stage.

Accordingly, in the surgery for cancer in the early stage, determination can be made whether or not the cancer has spread to lymph nodes by detecting the sentinel lymph nodes during the excision surgery for the cancer, performing biopsy, and performing a quick pathological examination. In the event that metastasis of cancer to the sentinel lymph nodes has not occurred, there is no need to perform excision of the remaining lymph nodes in the surgery for cancer in the early stage. On the other hand, in the event that metastasis of cancer to the sentinel lymph nodes has occurred, excision is performed for multiple lymph nodes near the affected portion in the surgery, based upon the state of metastasis of cancer in the early stage.

With the surgery for cancer in the early stage, the above-described sentinel node navigation surgery is performed, and thus excessive excision of lymph nodes wherein metastasis of cancer has not occurred is not performed for the patient without metastasis of cancer to lymph nodes, thereby reducing the burden on the patient. Furthermore, the sentinel node navigation surgery is applied not only to surgery for breast cancer, for example, but also to laparotomy for the stomach or the like, or surgery using a peritoneoscope.

With regard to the sentinel node navigation surgery, there is a great demand for a detecting device for easily and precisely detecting the sentinel lymph nodes.

Examples of the detecting devices include arrangements disclosed in Japanese Unexamined Patent Application Publication No. 2001-299676, Japanese Unexamined Patent Application Publication No. 9-189770, Japanese Unexamined Patent Application Publication No. 10-96782, U.S. Pat. No. 6,205,352, and the like.

Furthermore, in recent years, a SQUID fluxmeter employing a superconducting quantum interference device (which will be abbreviated to “SQUID” hereafter) is applied to various fields. The SQUID can detect magnetic flux around 1/1,000,000,000 of the terrestrial magnetism with high sensitivity.

In recent years, with regard to the SQUID, a high temperature superconductivity SQUID which can be used under refrigeration at the temperature (77.3 K: −196° C.) of liquid nitrogen has been put to practical use.

Thus, proposals have been made regarding the detecting devices employing the high temperature superconductivity SQUID as described in the Journal of the Japan Biomagnetism and Bioelectromagnetics Society special issue (Vol. 15, No. 1, 2002, No. 17, pp. 31-32).

### SUMMARY OF THE INVENTION

A magnetic fluid detecting apparatus for identifying sentinel lymph nodes according to the present invention comprises a single or multiple magnets for exciting magnetic fluid accumulated within the subject, and multiple magnetic sensors for detecting the distortion of the local magnetic distribution due to the magnetic fluid excited by the magnets, wherein any of the magnet, the combination of the magnet and the multiple magnetic sensors, and the combination of the magnet, the multiple magnetic sensors, and a preamplifier for amplifying the outputs from the multiple magnetic sensors, is vibrated or rotated, and the difference between the outputs from the multiple magnetic sensors is obtained and is subjected to demodulation, thereby detecting the magnetic fluid.

Furthermore, another magnetic fluid detecting apparatus for identifying sentinel lymph nodes according to the present invention comprises a single or multiple electromagnets for exciting magnetic fluid accumulated within the subject, and multiple magnetic sensors for detecting the distortion of the local magnetic distribution due to the magnetic fluid excited by the multiple electromagnets, wherein the electromagnets are driven by AC current, and the difference between the outputs from the multiple magnetic sensors is obtained and is subjected to demodulation, thereby detecting the magnetic fluid.

Furthermore, another magnetic fluid detecting apparatus for identifying sentinel lymph nodes according to the present invention comprises a single or multiple magnets for exciting magnetic fluid accumulated within the subject, multiple magnetic sensors for detecting the distortion of the local magnetic distribution due to the magnetic fluid excited by the magnets, and a variable offset unit for offsetting the difference between the outputs from the multiple magnetic sensors, wherein any of the magnet, the combination of the magnet and the multiple magnetic sensors, and the combination of the magnet, the multiple magnetic sensors, and a preamplifier for amplifying the outputs from the multiple magnetic sensors, is vibrated or rotated, and the difference between the outputs from the multiple magnetic sensors is obtained and is subjected to demodulation, thereby detecting the magnetic fluid, and the variable offset unit adjusts the amount of the offset based upon the output from the preamplifier.

Furthermore, another magnetic fluid detecting apparatus for identifying sentinel lymph nodes according to the present invention comprises a single or multiple magnets for exciting magnetic fluid accumulated within the subject, and multiple magnetic sensors for detecting the distortion of the local magnetic distribution due to the magnetic fluid excited by the magnets, wherein any of the magnet, the combination of the magnet and the multiple magnetic sensors, and the combination of the magnet, the multiple magnetic sensors, and a preamplifier for amplifying the outputs from the multiple magnetic sensors, is vibrated in the direction parallel to a line or a plane including the multiple magnetic sensors, and the difference between the outputs from the multiple magnetic sensors is obtained and is subjected to demodulation.

### BRIEF DESCRIPTION OF THE DRAWINGS

FIG. 1 through FIG. 9 relate to a first embodiment of the present invention, wherein FIG. 1 is an overall configuration diagram which illustrates a magnetic fluid detecting apparatus for identifying sentinel lymph nodes according to the first embodiment;

FIG. 2A is an explanatory diagram which shows the principle of detection of the external magnetic field (magnetic flux density thereof), and is a conceptual diagram which shows the state of the exciting magnet and the magnetic sensors in a case that there is no substance (magnetic fluid) with magnetic permeability higher than the surroundings;

FIG. 2B is a conceptual diagram which shows the state of the exciting magnet and the magnetic sensors in a case that there is a substance (magnetic fluid) with magnetic permeability higher than the surroundings;

FIG. 3A is an explanatory diagram which shows detection action of the magnetic sensors, and is a conceptual diagram which shows detection action of the magnetic sensors in a case that there is no external magnetic field (magnetic flux density thereof) B;

FIG. 3B is a conceptual diagram which shows detection action of the magnetic sensors in a case that there is an external magnetic field (magnetic flux density thereof) B;

FIG. 4A is a conceptual diagram which shows detection action of the magnetic sensors in a case that there is an external magnetic field (magnetic flux density thereof) B;

FIG. 4B is a schematic diagram which shows a circuit of FIG. 4A;

FIG. 4C is a circuit block diagram which shows a circuit configuration including a four-terminal bridge formed of a combination of two MR sensors and fixed resistors R3 and R4, an amplifier for performing differential amplification for the outputs from the four-terminal bridge, and an AC differential amplifier;

FIG. 5 is a circuit block diagram which shows a circuit configuration of the magnetic fluid detecting apparatus according to the first embodiment;

FIG. 6 is an explanatory diagram which illustrates a first modification of the probe shown in FIG. 1;

FIG. 7 is a schematic diagram which illustrates electrodes which can be rotated;

FIG. 8 is an explanatory diagram which shows a second modification of the probe shown in FIG. 1;

FIG. 9 is an explanatory diagram which shows a third modification of the probe shown in FIG. 1;

FIG. 10 through FIG. 17 relate to a second embodiment of the present invention, wherein FIG. 10 illustrates a probe forming a magnetic fluid detecting apparatus for identifying sentinel lymph nodes according to the second embodiment;

FIG. 11 is an explanatory diagram for describing the exciting electromagnet shown in FIG. 10;

FIG. 12 is an explanatory diagram for describing an exciting electromagnet having a configuration wherein an exciting coil has been wound onto a U-shaped exciting magnet, employed instead of the exciting electromagnet shown in FIG. 11;

FIG. 13 is an explanatory diagram for describing a first modification of the probe shown in FIG. 10;

FIG. 14A is an explanatory diagram for describing an arrangement wherein MR sensors are employed as magnetic sensors connected one to another in serial;

FIG. 14B is an explanatory diagram for describing an arrangement wherein MR sensors are connected one to another in parallel;

FIG. 15 is a circuit block diagram which shows a circuit configuration of the magnetic fluid detecting apparatus according to the second embodiment;

FIG. 16A is an explanatory diagram for describing an arrangement wherein the magnetic sensors and the exciting electromagnet are formed of a thin film using the semiconductor process, and is a front view which illustrates a silicon substrate where thin-film magnetic sensors and a thin-film coil have been formed;

FIG. 16B is a side view of FIG. 16A;

FIG. 17 is an explanatory diagram for describing a second modification of the probe shown in FIG. 10;

FIG. 18 through FIG. 24 relate to a third embodiment of the present invention, wherein FIG. 18 is an explanatory diagram for describing a probe forming a magnetic fluid detecting apparatus for identifying sentinel lymph nodes according to the third embodiment;

FIG. 19 is an explanatory diagram for describing the exciting electromagnet shown in FIG. 18;

FIG. 20 is a chart which shows the magnetic flux density of the exciting magnetic field formed by the exciting electromagnet shown in FIG. 19;

FIG. 21 is a circuit block diagram which shows a circuit configuration of the magnetic fluid detecting apparatus according to the third embodiment;

FIG. 22 is a chart which shows the change in the magnetic flux density over time formed by the exciting electromagnet AC driven by the exciting coil driving circuit shown in FIG. 20;

FIG. 23 is a chart which shows the differential output detected by the magnetic sensors in a case that there is magnetic fluid;

FIG. 24 is an explanatory diagram for describing an arrangement including monitor magnetic sensors;

FIG. 25 through FIG. 34 relate to a fourth embodiment of the present invention, wherein FIG. 25 is an overall configuration diagram which illustrates a magnetic fluid detecting apparatus for identifying sentinel lymph nodes according to the fourth embodiment;

FIG. 26 is a circuit block diagram which shows a circuit configuration of a control device;

FIG. 27 is a circuit block diagram which shows a circuit configuration of a sensor unit according to the present embodiment;

FIG. 28 is a schematic diagram which illustrates the sensor unit detecting magnetic fluid accumulated in a sentinel lymph node;

FIG. 29 is a flowchart which shows control of a P.C.;

FIG. 30 is a schematic diagram which illustrates the probe with the attitude being changed within the terrestrial magnetism;

FIG. 31 is a circuit block diagram of the sensor unit having an assumed circuit configuration;

FIG. 32 is a chart which shows the output Vout from the sensor unit shown in FIG. 31;

FIG. 33 is a circuit block diagram which shows a first modification of the sensor unit;

FIG. 34 is a circuit block diagram which shows a second modification of the sensor unit;

FIGS. 35 and 36 relate to a fifth embodiment of the present invention, wherein FIG. 35 is an overall configuration diagram which illustrates a magnetic fluid detecting apparatus for identifying sentinel lymph nodes;

FIG. 36 is a circuit block diagram which shows a circuit configuration of the control device shown in FIG. 35;

FIG. 37 through FIG. 40 relate to a sixth embodiment of the present invention, wherein FIG. 37 is an explanatory diagram for describing a probe forming a magnetic fluid detecting apparatus for identifying sentinel lymph nodes according to the sixth embodiment;

FIG. 38A is a schematic diagram which illustrates minute vibration of the sensor unit being performed in the direction parallel to a line including two MR sensors;

FIG. 38B is a chart which shows signals from the MR sensors of the sensor unit shown in FIG. 38A;

FIG. 39 is a chart which shows signals obtained from the sensor unit shown in FIG. 38A, which have been subjected to the Fourier transformation;

FIG. 40A is a schematic diagram which illustrates minute vibration of the sensor unit being performed in the direction orthogonal to a line including two MR sensors, and in the longitudinal direction thereof including a magnet; and

FIG. 40B is a chart which shows signals from the MR sensors of the sensor unit shown in FIG. 40A.

### DETAILED DESCRIPTION OF PREFERRED EMBODIMENTS

Description will be made below regarding embodiments according to the present invention with reference to the drawings.

(First Embodiment)

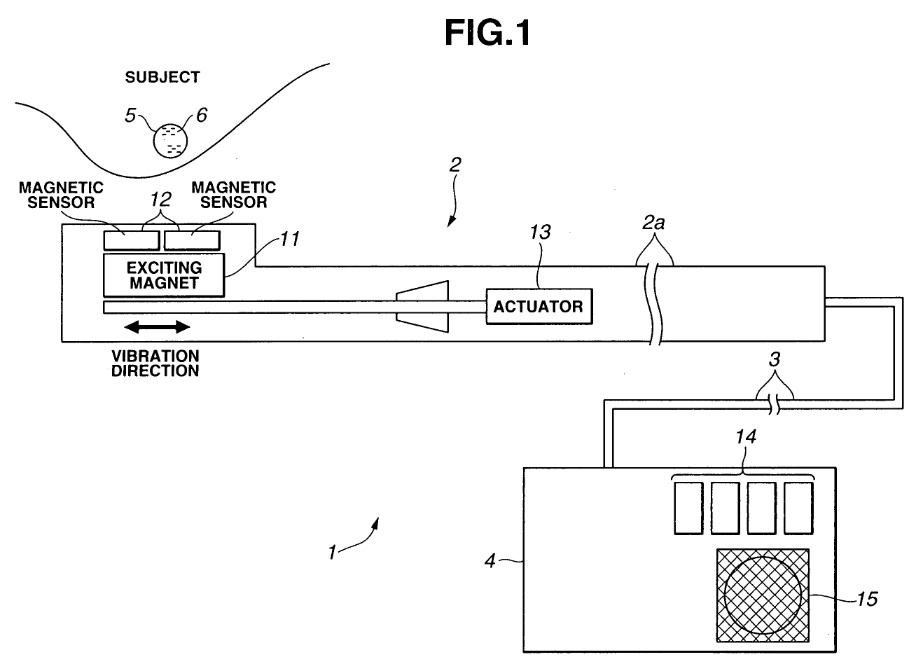

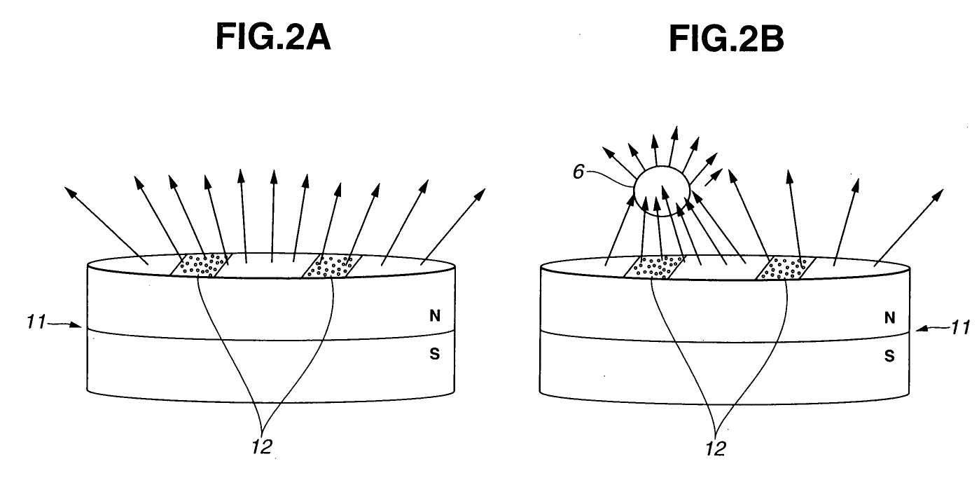

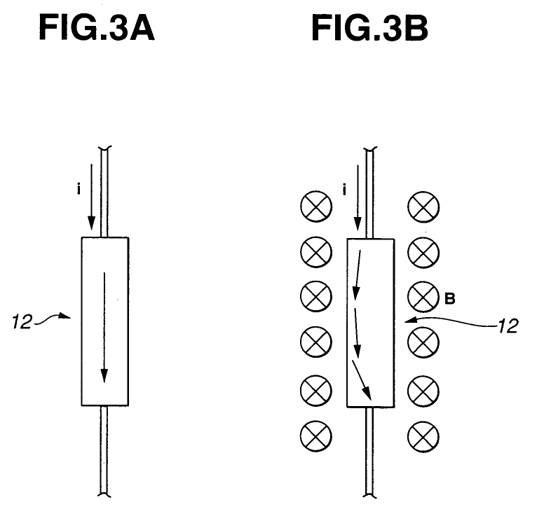

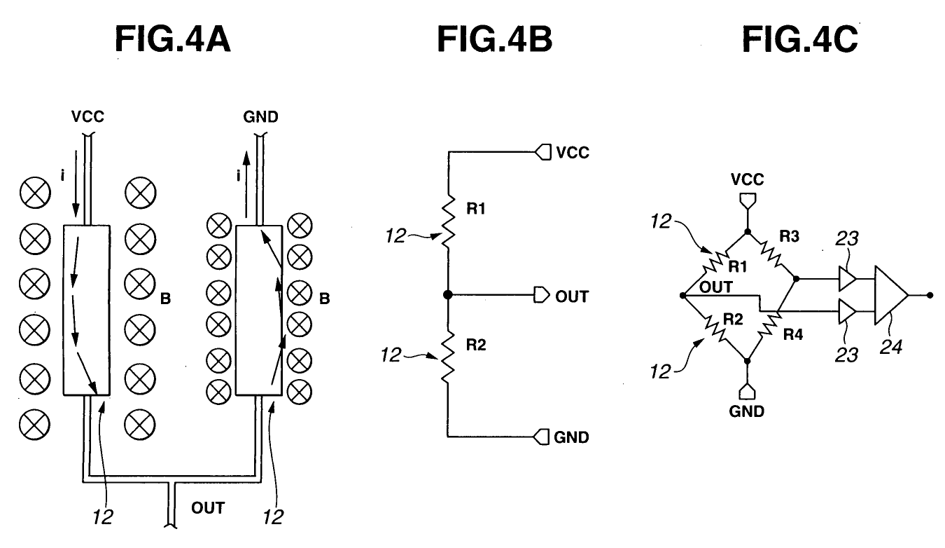

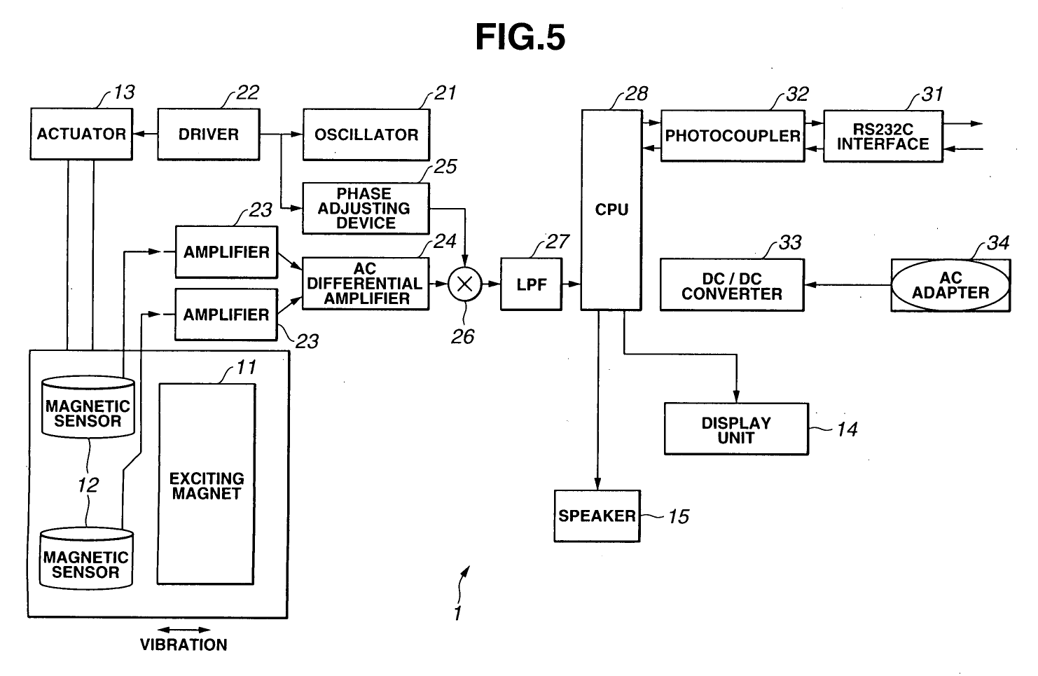

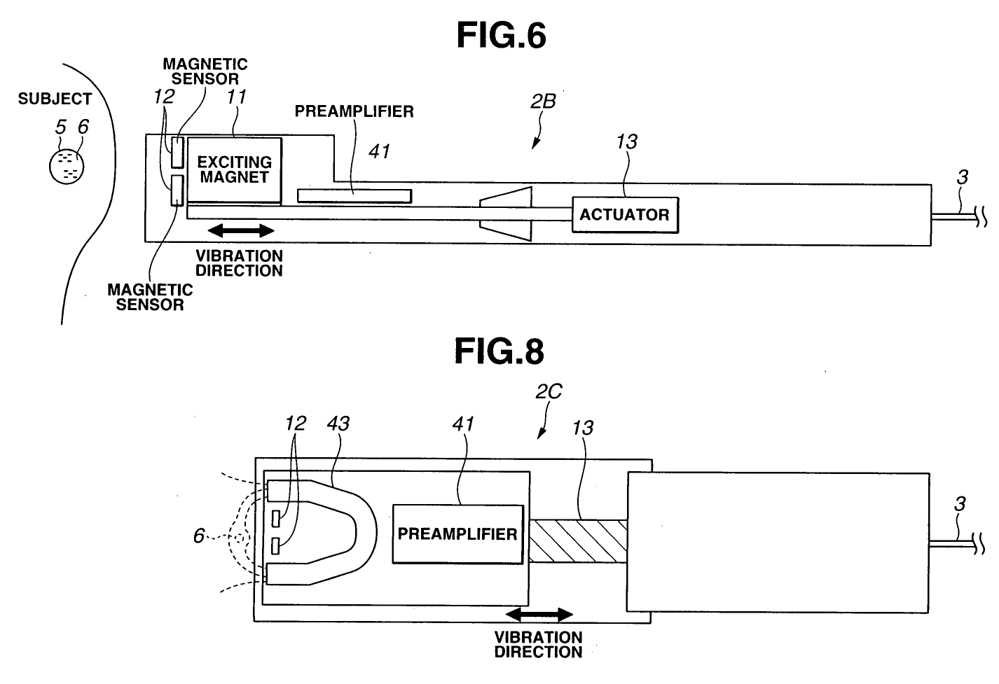

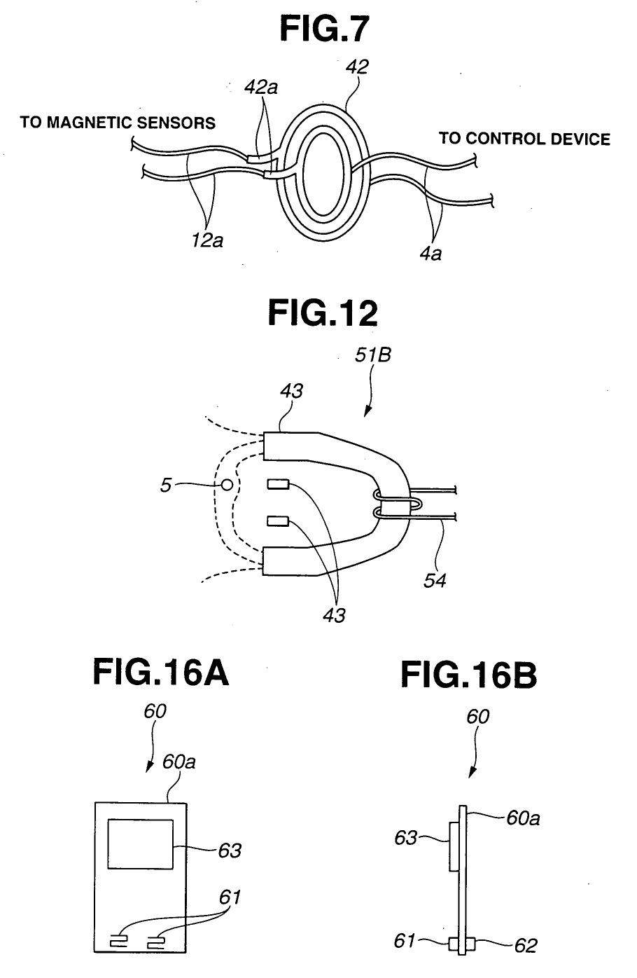

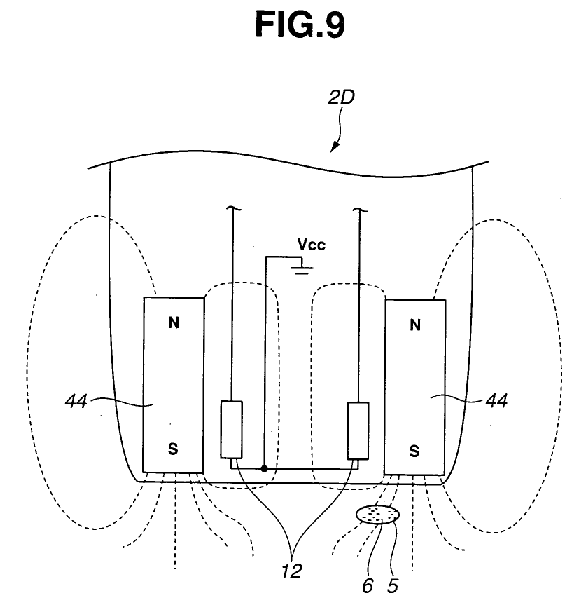

FIGS. 1 through 9 relate to a first embodiment according to the present invention, wherein FIG. 1 is an overall configuration diagram which illustrates a magnetic fluid detecting apparatus for identifying sentinel lymph nodes according to the first embodiment, FIG. 2A is an explanatory diagram for describing the principle of detecting the external magnetic field (magnetic-flux density) and in greater detail, is a conceptual diagram which illustrates the state of an exciting magnet and magnetic sensors without a substance (magnetic fluid) with magnetic permeability higher than the surroundings, FIG. 2B is a conceptual diagram which illustrates the state of an exciting magnet and magnetic sensors with a substance (magnetic fluid) with magnetic permeability higher than the surroundings, FIG. 3A is an explanatory diagram for describing detection action of the magnetic sensor, and in greater detail, is a conceptual diagram which illustrates detection action of the magnetic sensors without the external magnetic field (magnetic-flux density) B therearound, FIG. 3B is a conceptual diagram which illustrates detection action of the magnetic sensors with the external magnetic field (magnetic-flux density) B therearound, FIG. 4A is an explanatory diagram for describing an arrangement employing two magnetic sensors shown in FIG. 3A, and is a conceptual diagram which shows detection action of the magnetic sensors with the external magnetic field (magnetic-flux density) B, FIG. 4B is a schematic diagram which illustrates a circuit of the arrangement shown in FIG. 4A, FIG. 4C is a circuit block diagram which shows a circuit configuration made up of a four terminal bridge formed of a combination of two MR sensors and two fixed resistors R3 and R4, an amplifier for performing differential amplification for the output from the four-terminal bridge, and an AC differential amplifier, FIG. 5 is a circuit block diagram which shows the circuit configuration of the magnetic fluid detecting apparatus according to the first embodiment, FIG. 6 is an explanatory diagram which illustrates a probe according to a first modification of the probe shown in FIG. 1, FIG. 7 is a schematic diagram which illustrates a slip ring used for the probe, FIG. 8 is an explanatory diagram for describing a probe according to a second modification for the probe shown in FIG. 1, and FIG. 9 is an explanatory diagram for describing a probe according to a third modification for the probe shown in FIG. 1.

As shown in FIG. 1, a magnetic fluid detecting apparatus 1 for identifying sentinel lymph nodes according to the first embodiment of the present invention comprises a probe 2 which can be inserted into the body cavities and includes an exciting magnet and magnetic sensors described later on the tip thereof, and a control device 4 for controlling the probe 2, connected to the probe 2 through a connecting cable 3.

The probe 2 includes an exciting magnet 11 for exciting magnetic fluid 6 accumulated in a sentinel lymph node 5 within the subject across a space near the probe, and two magnetic sensors 12 for detecting the distortion of the local magnetic field distribution (the change in the magnetic flux density) due to the excited magnetic fluid 6, whereby the sentinel lymph node 5 is identified.

Furthermore, the above-described probe 2 includes an actuator 13 for vibrating the exciting magnet 11 and the magnetic sensors 12 in the direction of the longitudinal axis thereof.

Thus, the magnetic fluid detecting apparatus 1 modulates the local magnetic field from the magnetic fluid 6, excited by the exciting magnet 11, detects the distortion of the local magnetic field distribution (the change in the magnetic flux density) with the two magnetic sensors 12, obtains the difference between the outputs from the two magnetic sensors 12, and furthermore performs demodulation, thereby removing magnetic noise with frequencies other than the modulation frequency due to the terrestrial magnetism or other electric devices.

Furthermore, the control device 4 includes a display unit 14 for displaying the change in the magnetic flux density detected by the magnetic sensors 12, made up of LEDs (Light Emitting Diodes), a LCD (Liquid Crystal Display), or the like, and a speaker 15 for notifying the change in the magnetic flux density by audio, on the front panel thereof.

Note that the probe 2 has a casing 2a formed of non-magnetic material, and has a waterproof configuration.

First, description will be made regarding the principle of the external magnetic field (magnetic flux density) with reference to FIGS. 2A and 2B.

As shown in FIG. 2A, the two magnetic sensors 12 have a configuration wherein measurement is performed for the spatial gradient of the magnetic field distribution (magnetic flux density) near the probe excited by the exciting magnet 11. Here, let us say that a substance is situated in the region of interest in the subject, with magnetic permeability higher than the surroundings such as the magnetic fluid 6 or the like, as shown in FIG. 2B. In this case, local distortion occurs for the magnetic field applied by the exciting magnet 11 due to the magnetic field being absorbed into the perimeter of the high-magnetic permeability substance, thereby effecting the change in the spatial gradient of the magnetic field distribution (magnetic flux density). Note that the arrows denote lines of magnetic force in FIGS. 2A and 2B.

The spatial gradient of the magnetic field distribution formed due to the high-permeability substance is detected by obtaining the difference between the outputs from the two magnetic sensors 12.

In the event that there is no high-permeability substance therearound, the spatial gradient of the magnetic field distribution is not formed, whereby the difference between the outputs from the two magnetic sensors 12 becomes zero. Thus, the two magnetic sensors 12 can detect the presence or absence of the substance with permeability higher than the surroundings.

Next, description will be made regarding an MR sensor (magnetic resistance sensor) which is an example of the magnetic sensors 12 with reference to FIGS. 3A, 3B, 4A, 4B, and 4C.

FIG. 3A shows the current path length wherein current flows within a conductor at the time of applying current i to the MR sensor, and FIG. 3B shows the current path length wherein current flows within a conductor at the time of applying current i to the MR sensor in the event that the external magnetic field B is applied in the direction orthogonal to the drawing.

At the time of applying the current i to the MR sensor, in a case of applying the external magnetic field B in the direction orthogonal to the drawing (FIG. 3B), the current path length wherein the current flows within a conductor becomes long as compared with in a case that the external magnetic field B is not applied in the direction orthogonal to the drawing (FIG. 3A). Accordingly, upon the external magnetic field B being applied, the resistance value of the MR sensor becomes great.

With the present embodiment, a combination of at least two MR sensors described above is employed as shown in FIGS. 4A and 4B. Upon the current i flowing from Vcc to GND, an electric potential corresponding to the ratio between the resistance values of the two MR sensors (magnetic sensors 12) occurs on the output side (OUT). In the event that the external magnetic field is applied to the two MR sensors (magnetic sensors 12) with the same magnitude, the resistance values of the two sensors become the same value, and accordingly, the electric potential on the output side (OUT) does not change.

On the other hand, in the event that the magnetic field is applied to the two MR sensors (magnetic sensors 12) with different magnitudes, the resistance values of the two MR sensors (magnetic sensors 12) become different values, and accordingly, the electric potential on the output side (OUT) changes. That is to say, the electric potential on the output side (OUT) changes in proportion to the degree of gradient of the magnetic field. This means that the difference between the outputs from the two MR sensors (magnetic sensors 12) is obtained.

In actual practice, a four-terminal bridge formed of the two MR sensors (magnetic sensors 12) is employed as shown in FIG. 4C in order to efficiently measure the spatial gradient of the magnetic field distribution.

As shown in FIG. 4C, the four-terminal bridge is formed of the two MR sensors (magnetic sensors 12) and fixed resistors R3 and R4. Furthermore, the four-terminal bridge has a configuration wherein the bridge output is subjected to differential amplification.

The principle of the differential amplification in the four-terminal bridge is the same as with in FIGS. 4A and 4B described above. That is to say, the bridge output from the four-terminal bridge changes due to the change in the resistance ratio between the two MR sensors (magnetic sensors 12). Subsequently, the bridge outputs from the four-terminal bridge are amplified by amplifiers 23, difference between the outputs is obtained and amplified by an AC differential amplifier 24, thereby obtaining the gradient of the external magnetic field.

Next, description will be made regarding the circuit configuration of the magnetic fluid detecting apparatus 1 with reference to FIG. 5.

The magnetic fluid detecting apparatus 1 comprises an oscillator 21 for generating signals with a predetermined oscillating frequency, a driver 22 for driving the actuator 13 according to the signals with the predetermined oscillating frequency generated from the oscillator 21, amplifiers 23 for amplifying the outputs from the magnetic sensors 12, an AC differential amplifier 24 for obtaining the difference between the outputs from the amplifiers 23 and amplifying the difference, a phase adjusting device 25 for adjusting the phase of the output from the AC differential amplifier 24, synchronously with the frequency of the oscillator 21, a multiplier 26 for multiplying the output from the AC differential amplifier 24 by the signal with the phase adjusted by the phase adjusting device 25, for removing noise components, a low pass filter (LPF) 27 for removing high-frequency components from the output from the multiplier 26, thereby obtaining amplitude components, and a CPU (Central Processing Unit) 28 for driving the display unit 14 and the speaker 15 by converting the change in the external magnetic field (magnetic flux density) B into numerical data based upon the output from the LPF 27. Note that the phase adjusting unit 25, the multiplier 26, and the LPF 27, have the same configuration as with a lock-in amplifier, which is a narrow-band bandpass filter.

Furthermore, the magnetic fluid detecting apparatus 1 communicates with a PC, a printer, or the like, through an unshown RS232C interface via a photocoupler 32, thereby enabling storage of data, printing of data, or the like. Note that the RS232C is connected to the magnetic fluid detecting apparatus with an RS232C interface 31.

Furthermore, the magnetic fluid detecting apparatus 1 includes an AC adapter 34 and a DC/DC converter 33 for generating voltage used for the apparatus, and supplying the voltage to each unit thereof.

The magnetic fluid detecting apparatus 1 having such a configuration is used for detecting the magnetic fluid 6 accumulated in the sentinel lymph node 5 within the subject so as to identify the sentinel lymph node 5.

First, the surgeon inserts an unshown puncture needle into the lower layer of the affected portion of the subject, and locally injects the magnetic fluid 6 near the affected portion. As a result, the magnetic fluid 6 injected around the affected portion moves from the injection portion to the lymph vessels, reaches the sentinel lymph node 5 after five to fifteen minutes, and is accumulated in the sentinel lymph node 5.

Subsequently, the surgeon uses the probe 2 of the magnetic fluid detecting apparatus 1 by inserting the probe 2 into the treatment-tool inserting channel of an unshown endoscope, or by surgically inserting the probe into the body cavity through an unshown trocar, for example. The magnetic fluid 6 accumulated in the sentinel lymph node 5 is detected while the surgeon moves the tip of the probe 2 as to the portion near the affected portion of the patient.

At this time, with the probe 2, the exciting magnet 11 excites the space near the probe, and the magnetic sensors 12 measure the spatial gradient of the magnetic field distribution (magnetic flux density). At this time, in the event that the magnetic fluid 6 is situated near the affected portion of the patient, the magnetic filed applied by the exciting magnet 11 is absorbed into the perimeter of the magnetic fluid 6, and accordingly, local distortion of the magnetic field distribution occurs, whereby the change in the spatial gradient of the magnetic field distribution (magnetic flux density) occurs.

At this time, with the probe 2, the actuator 13 is driven according to the signals with a predetermined oscillating frequency, whereby the exciting magnet 11 and the magnetic sensors 12 are vibrated in the longitudinal direction. As a result, the local magnetic field due to the magnetic fluid 6 is subjected to modulation due to the vibration of the exciting magnet 11.

Subsequently, the distortion of the local magnetic field distribution (the change in magnetic flux density) due to the magnetic fluid 6, which has been subjected to modulation, is detected by the two magnetic sensors 12. The outputs from the two magnetic sensors 12 are each amplified by the amplifiers 23, and the difference between the outputs is obtained and amplified by the AC differential amplifier 24.

In this case, the magnetic sensors 12 detect the local magnetic field (the horizontal component of the magnetic field) while vibrating in the longitudinal direction as to the distortion of the local magnetic field distribution (the change in the magnetic flux density) near the magnetic fluid 6. Note that the change in the detected amplitude is dependent upon the amount of the magnetic fluid 6.

The output from the AC differential amplifier 24 is multiplied by the multiplier 26, by the phase adjusted by the phase adjusting device 25, whereby noise components are removed. The output from the multiplier 26 is subjected to removal of high-frequency components by the LPF 27, whereby the amplitude components are obtained. Thus, the output from the AC differential amplifier 24 is demodulated.

The signal is output to the CPU 28. The CPU 28 converts the change in the magnetic flux density into the numerical data so as to drive the display unit 14 and the speaker 15. The display unit 14 displays the change in the magnetic flux density with an indicator or in numbers. In this case, the display unit 14 has a configuration wherein the closer the tip of the probe 2 moves to the magnetic fluid 6, the greater the amplitude of the indicator or the displayed numerical value is, and conversely, the farther the tip of the probe 2 moves from the magnetic fluid 6, the smaller the amplitude of the indicator or the displayed numerical value is.

Furthermore, the speaker 15 generates sound corresponding to the change in the magnetic flux density. In this case, the closer the tip of the probe 2 moves to the magnetic fluid 6, the greater the sound generated by the speaker 15 is, and conversely, the farther the tip of the probe 2 moves from the magnetic fluid 6, the smaller the sound generated by the speaker 15 is. Or, the speaker 15 generates sound with the frequency in proportion to the distance between the probe 2 and the magnetic fluid 6.

Thus, the magnetic fluid detecting apparatus 1 according to the present embodiment can detect the precise position of the magnetic fluid 6 accumulated in the sentinel lymph node 5 with excellent operability, thereby enabling identification of the position of the sentinel lymph node 5.

As can be understood from the above description, the magnetic fluid detecting apparatus 1 according to the present embodiment detects the gradient of the magnetic field formed by the distortion of the magnetic field applied by the exiting magnet 11 due to the magnetic fluid 6, thereby enabling identification of the sentinel lymph node 5 where the magnetic fluid 6 has been accumulated, even without using a high-sensitivity sensor such as a SQUID sensor. Furthermore, the magnetic fluid detecting apparatus 1 according to the present embodiment performs modulation by vibrating the sensor unit (two magnetic sensors 12) and subsequently, demodulation is performed for the modulated signals, thereby removing magnetic noise due to other external electric devices or the like.

In this case, the amplitude of the vibration of the sensor unit (two magnetic sensors 12) and the exciting magnet 11 is small, and accordingly, the magnetic fluid detecting apparatus 1 according to the present embodiment is not influenced by the spatial distribution of the magnetic noise due to external electric devices or the like, but is influenced only by the local spatial distribution of the magnetic field formed by the magnetic fluid 6.

That is to say, the magnetic fluid detecting apparatus 1 according to the present embodiment converts only the amount of the distortion of the magnetic field due to the magnetic fluid 6 into modulated signals so as to be detected, thereby obtaining the demodulated signals corresponding to the influence of the magnetic fluid, and thereby removing magnetic noise and the like.

Thus, the magnetic fluid detecting method 1 according to the present embodiment can identify the precise position of the sentinel lymph node 5 with small in size, excellent operability and low costs.

Note that the probe may have a configuration as shown in FIG. 6.

As shown in FIG. 6, a probe 2B has a configuration wherein magnetic sensors 12 are provided on the tip side of the exciting magnet 11. In this case, with the magnetic fluid detecting apparatus, the surgeon can search the sentinel lymph node 5 with the probe 2B erected within the body cavity, thereby improving the operability within a narrow space.

The probe 2B has a configuration wherein the preamplifier unit (preamplifier 41) is vibrated along with the magnetic sensors 12 at the same time.

In the event that MR sensors are employed for the magnetic sensors 12, the probe measures the change in the resistance due to the magnetic field, and accordingly, the change in the resistance due to the change in the shape of a lead wire between the MR sensor and an amplifier prevents precise measurement of the change in the magnetic field.

Accordingly, with the probe 2B according to the present modification, the above-described problem can be prevented by vibrating a preamplifier 41 along with the magnetic sensors 12, thereby enabling precise measurement of the change in the magnetic field. Note that the preamplifier 41 may include only the amplifier 23, or may further include the AC differential amplifier 24.

Furthermore, the probe may have a configuration wherein the exciting magnet 11 and the magnetic sensors 12 can be rotated, which is not shown in the drawings. In this case, the probe includes a slip ring 42 for electric connection between the magnetic sensors 12 and the control device 4, as shown in FIG. 7.

As shown in FIG. 7, the slip ring 42 is in contact with an electrode brushes 42a connected to signal lines 12a to the side of the magnetic sensors 12 on the tip side, and is connected to signal lines 4a to the side of the control device 4 on the base side. With the probe 2, use of the slip ring 42 enables preventing the signal lines 12a to the side of the magnetic sensors 12 from being damaged or disconnected by being twisted due to rotation of the actuator 13.

Furthermore, the probe may have a configuration including a U-shaped exciting magnet as shown in FIG. 8.

As shown in FIG. 8, a probe 2C has a configuration including a U-shaped exciting magnet 43 as the exciting magnet 11. In the event that the magnetic field is applied with too great a magnitude, saturation of the magnetic sensors 12 (MR sensors) occurs, leading to difficulties in detection of the magnetic field.

The U-shaped exciting magnet 43 forms the magnetic field so that the magnetic field is emitted from the one end thereof and is absorbed into the other end thereof. Furthermore, the magnetic sensors 12 are disposed within the U-shaped exciting magnet 43 so that the magnetic sensors 12 are not influenced by the magnetic field formed by the U-shaped exciting magnet 43. Specifically, the U-shaped exciting magnet 43 forms the magnetic field so as to be generally parallel as to the magnetic sensors 12. Note that the broken lines denote lines of magnetic force in the drawing.

Thus, the probe 2C can form a great magnetic field in the order of k (kilo) gauss for exciting the magnetic fluid 6 as compared with the exciting magnet 11 described in the above first embodiment, and furthermore, the magnetic field is formed with a small magnitude near the magnetic sensors 12.

Thus, the probe 2C can efficiently detect only the distortion of the local magnetic field distribution (the change in magnetic flux distribution) formed by the magnetic fluid 6, using the magnetic sensors 12. Note that the magnet employed for the exciting magnet 11 is not restricted to a U-shaped magnet, and a horseshoe-shaped magnet may be employed for the exciting magnet 11. Furthermore, the probe may have a configuration wherein multiple magnets are arrayed in the same direction of the N pole and the S pole as shown in FIG. 9.

As shown in FIG. 9, a probe 2D has a configuration wherein the exciting magnet 11 is formed of multiple exciting magnets 44 arrayed in the same direction of the N pole and the S pole, and the magnetic sensors 12 are disposed so that the magnetic sensors 12 are not influenced by the magnetic field formed by the exciting magnets 44. Note that the dotted lines denote lines of magnetic force in the drawing.

Thus, the probe 2D can form a great magnetic field for exciting the magnetic fluid 6 in the same way as with the above-described probe 2C, and furthermore, the magnetic field is formed with a small magnitude near the magnetic sensors 12. Thus, the probe 2D can efficiently detect only the distortion of the local magnetic field distribution (the change in magnetic flux distribution) formed by the magnetic fluid 6, using the magnetic sensors 12.

While description has been made in the present embodiment regarding an arrangement wherein MR sensors are employed for the magnetic sensors 12, an arrangement may be made wherein other magnetic sensors such as hole element sensors, GMR sensors, MI sensors, or the like, are employed for the magnetic sensors 12, as well. In a case of employing the MI sensors, the magnetic field causing saturation is small as compared with the MR sensors, and accordingly, the configuration such as probe 2C or 2D is particularly effective.

(Second Embodiment)

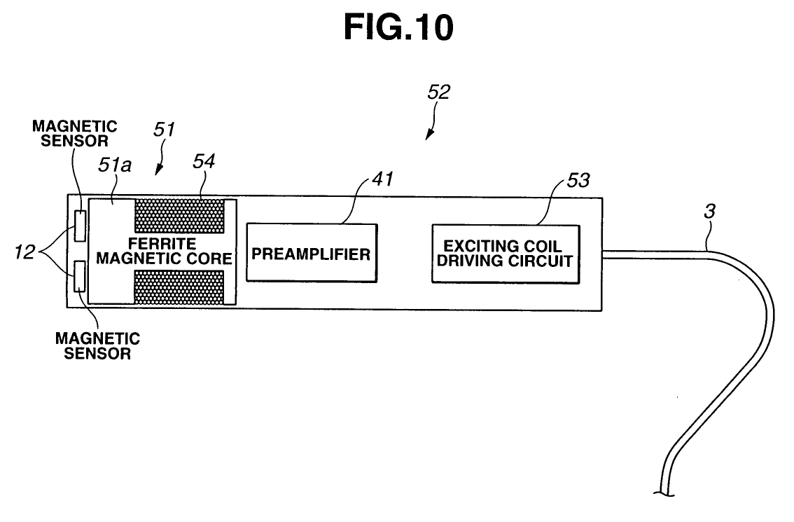

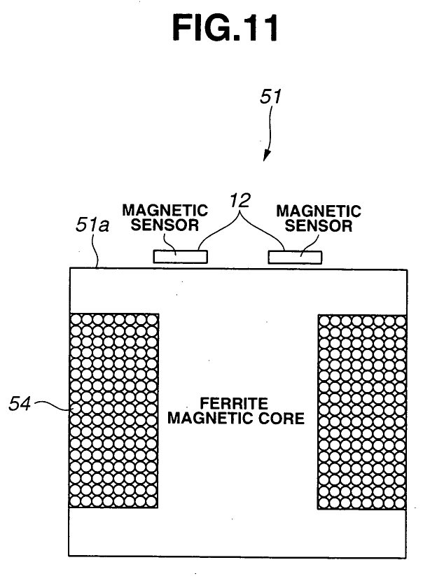

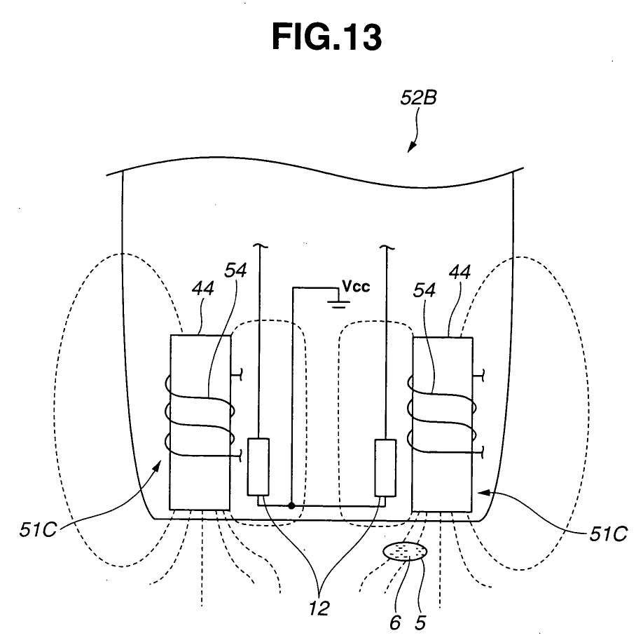

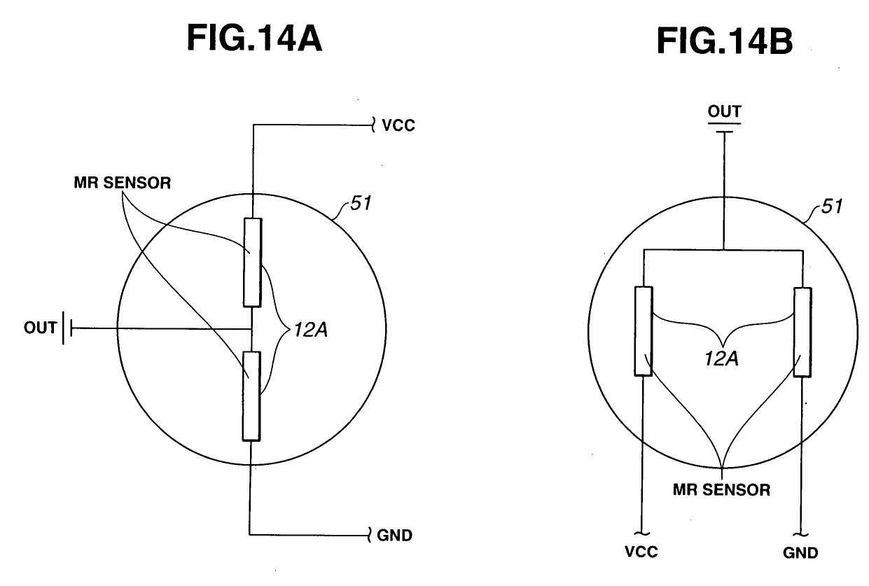

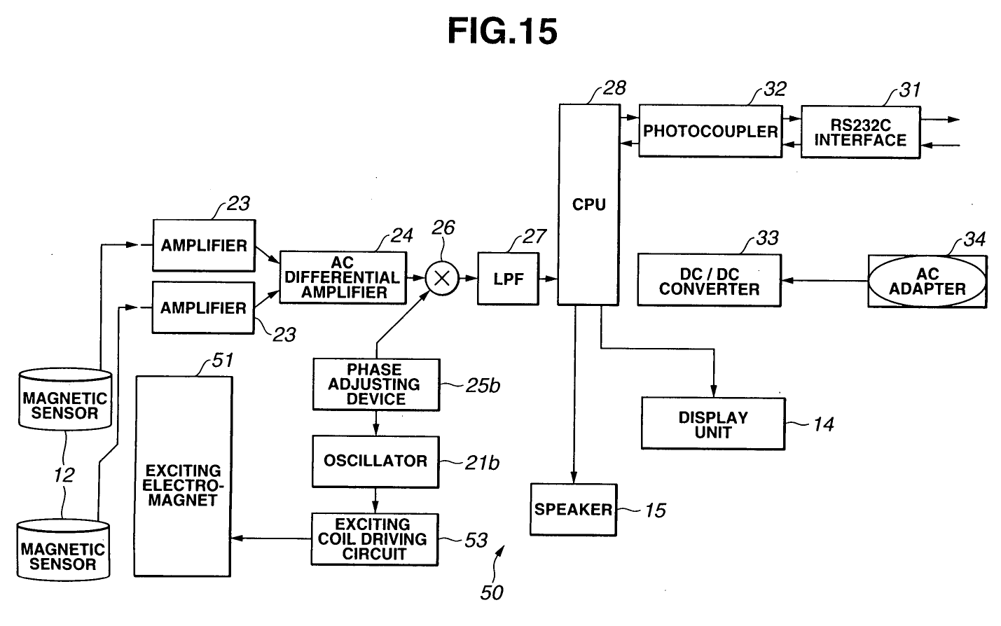

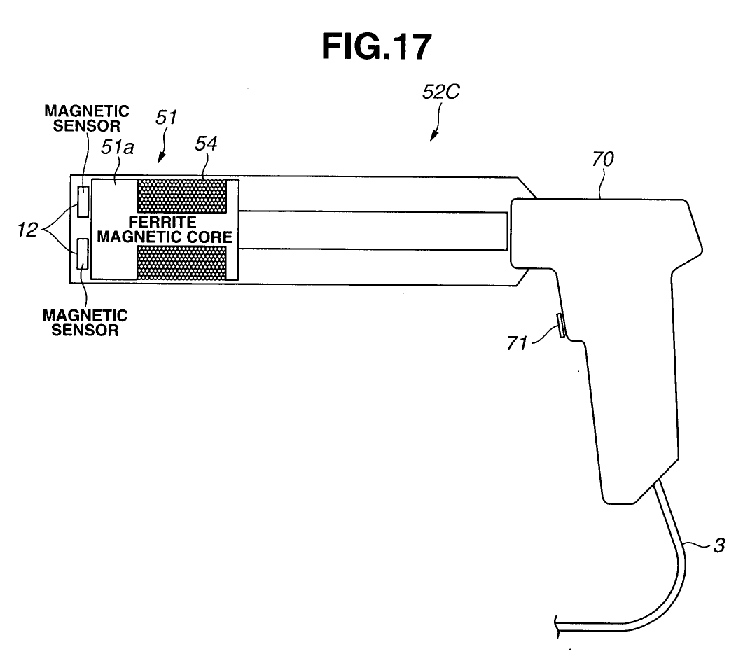

FIGS. 10 through 17 relate to a second embodiment according to the present invention, wherein FIG. 10 is an explanatory diagram which shows a probe forming a magnetic fluid detecting apparatus according to the second embodiment, FIG. 11 is an explanatory diagram which shows an exciting electromagnet shown in FIG. 10, FIG. 12 is an explanatory diagram which shows an exciting electromagnet formed of a U-shaped exciting magnet with an exciting coil wound thereon, instead of the exciting electromagnet shown in FIG. 11, FIG. 13 is an explanatory diagram which illustrates a first modification of the probe shown in FIG. 10, FIG. 14A is an explanatory diagram which shows an arrangement wherein MR sensors are employed for magnetic sensors, and is an explanatory diagram which shows an arrangement wherein the MR sensors are connected in serial, FIG. 14B is an explanatory diagram which shows an arrangement wherein MR sensors are connected one to another in parallel, FIG. 15 is a circuit block diagram which shows a circuit configuration of the magnetic fluid detecting apparatus according to the second embodiment, FIG. 16A is an explanatory diagram which shows an arrangement wherein the magnetic sensors and the exciting electromagnet are formed of thin films using the semiconductor process, and is a front view which illustrates a silicon substrate where thin-film magnetic sensors and a thin film coil have been formed, FIG. 16B is a side view of FIG. 16A, and FIG. 17 is an explanatory diagram which illustrates a second modification of the probe shown in FIG. 10.

The magnetic fluid detecting apparatus according to the second embodiment has a configuration wherein an exciting electromagnet is employed as an exciting magnet. The configuration other than the exciting magnet is generally the same as with the above-described first embodiment, so description will be omitted regarding the same configuration, and description will be made regarding the same components with the same reference numerals.

A magnetic fluid detecting apparatus 50 (see FIG. 15) according to the second embodiment includes a probe 52 having an exciting electromagnet 51 in the inside thereof on the tip side thereof, instead of the exciting magnet 11, as shown in FIG. 10. Furthermore, the magnetic fluid detecting apparatus 50 includes the preamplifier 41 in the inside of the probe 52 and an exciting coil driving circuit 53 for driving the exciting electromagnet 51. The exciting electromagnet 51 has a configuration wherein an exciting coil 54 is wound onto a ferrite magnetic core 51a as shown in FIG. 11.

Note that an arrangement may be made wherein a U-shaped exciting magnet is employed for the exciting electromagnet as shown in FIG. 12.

As shown in FIG. 12, an exciting electromagnet 51B has a configuration wherein the exciting coil 54 is wound onto the U-shaped exciting magnet 43 or an unshown horseshoe-shaped exciting magnet.

Thus, the magnetic fluid detecting apparatus 50 can form a great magnetic field in the order of k (kilo) gauss for exciting the magnetic fluid 6, and furthermore, the magnetic field is formed with a small magnitude near the magnetic sensors 12 in the same way as in FIG. 8 described above, thereby enabling efficient detection of only the distortion of the local magnetic field distribution (the change in magnetic flux distribution) formed by the magnetic fluid 6, using the magnetic sensors 12. Note that the magnet employed for the exciting magnet 51B is not restricted to a U-shaped magnet, and an unshown horseshoe-shaped magnet may be employed for the exciting magnet 51B. Furthermore, the exciting electromagnet may have a configuration wherein multiple magnets are arrayed in the same direction as with the N pole and the S pole as shown in FIG. 13.

As shown in FIG. 13, a probe 52B includes an exciting electromagnet 51C formed of exciting magnets 44 arrayed in the same direction as with the N pole and the S pole with the exciting coils 54 wound thereto.

Thus, the probe 52B can form a great magnetic field for exciting the magnetic fluid 6, and furthermore, the magnetic field is formed with a small magnitude near the magnetic sensors 12 in the same way as in FIG. 9 described above, thereby enabling efficient detection of only the distortion of the local magnetic field distribution (the change in magnetic flux distribution) formed by the magnetic fluid 6, using the magnetic sensors 12.

Now, examples of the layout of the magnetic sensors 12 are shown in FIGS. 14A and 14B. The magnetic sensors 12 employing MR sensors 12A detect the magnetic field orthogonal to the applied current, and accordingly, an arrangement may be made wherein the MR sensors 12A are arrayed in serial as shown in FIG. 14A, or an arrangement may be made wherein the MR sensors 12A are arrayed in parallel as shown in FIG. 14B.

Next, description will be made regarding a circuit configuration of the magnetic fluid detecting apparatus 50 according to the second embodiment with reference to FIG. 15.

The magnetic fluid detecting apparatus 50 includes a phase adjusting device 25b for adjusting the phase of the output from the AC differential amplifier 24, an oscillator 21b for generating signals with a predetermined oscillating frequency based upon the signals with the phase adjusted by the phase adjusting device 25b, and the exciting coil driving circuit 53, in addition to the circuit configuration of the magnetic fluid detecting apparatus 1 described in the above first embodiment. The exciting coil driving circuit 53 drives the exciting electromagnet 51 by applying an AC current to the exciting coil 54 with the oscillating frequency from the oscillator 21b.

More specifically, the exciting coil driving circuit 53 performs AC driving for alternating the direction of the excited magnetic field (the direction of the N pole and the S pole) based upon the oscillating frequency from the oscillator 21b.

The exciting electromagnet 51 generates an alternating magnetic field by the exciting coil 54 receiving supply of an AC current with a predetermined oscillating frequency from the exciting coil driving circuit 53, thereby exciting the magnetic fluid 6, as well as modulating the local magnetic field due to the excited magnetic fluid 6.

Note that circuit configurations other than the configuration described above are the same as in the description in the above first embodiment.

The magnetic fluid detecting apparatus 50 having such a configuration is used for identifying the sentinel lymph node 5 where the magnetic fluid 6 has been accumulated, in the same way as in the description in the above first embodiment.

That is to say, with the magnetic fluid detecting apparatus 50, the exciting electromagnet 51 excites a space near the probe, and the magnetic sensors 12 measure the spatial gradient of the magnetic field distribution (magnetic flux density). At this time, the probe 52 generates an alternating magnetic field by the exciting coil 54 of the exciting electromagnet 51 receiving supply of an AC current with a predetermined oscillating frequency.

As a result, the magnetic fluid 6 is excited, as well as the local magnetic field being modulated. The local magnetic field due to the magnetic fluid 6 subjected to modulation is detected by the two magnetic sensors 12. Note that the following operation is the same as in the description in the above first embodiment, so description thereof will be omitted.

As a result, the magnetic fluid detecting apparatus 50 according to the second embodiment has the same advantages as with the above-described first embodiment, and furthermore, moving components such as the actuator 13 or the like are not employed, and accordingly, the magnetic fluid detecting apparatus 50 has a simple configuration without vibration, thereby improving operability.

Note that the magnetic sensors 12 and the exciting electromagnet 51 may be formed of thin films using the semiconductor process as shown in FIGS. 16A and 16B.

As shown in FIGS. 16A and 16B, a silicon substrate 60 has a configuration wherein a thin-film magnetic sensors 61 and a thin-film coil 62 serving as an exciting coil are formed on a circuit on the substrate 60a. Furthermore, a MOS (Metal Oxide Semiconductor)—IC (Integrated Circuit) amplifier 63 can be formed on the silicon substrate 60 serving as a preamplifier, as well.

Thus, the probe can be formed with a smaller size.

Furthermore, the probe may have a configuration of a shape which the user can easily handle, as shown in FIG. 17.

As shown in FIG. 17, a probe 52C includes a holding portion 70 on the base side thereof. Furthermore, the holding portion 70 includes an operating switch 71, wherein upon the operating switch 71 being pressed, the detected value of the distortion of the local magnetic field distribution (the change in the magnetic flux density) is held. Thus, the user can narrow down several candidates for selecting the sentinel lymph node 5 by comparing between the values obtained by the probe 52C.

Note that the operating switch 71 may be provided for turning on or off the supplied current to the exciting coil 54.

(Third Embodiment)

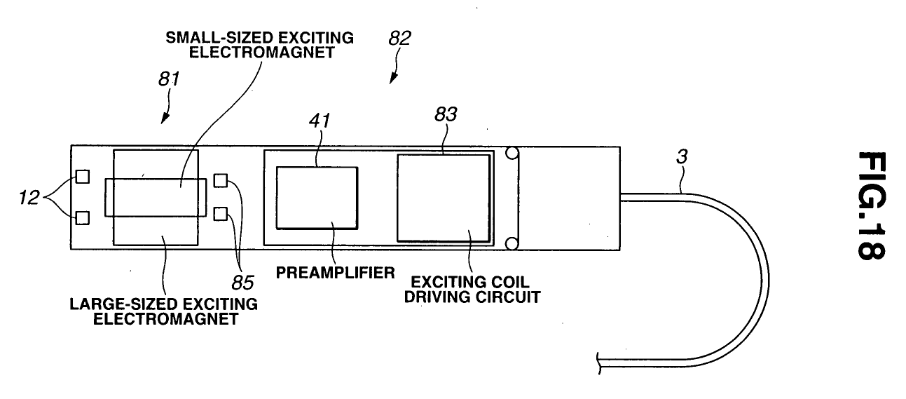

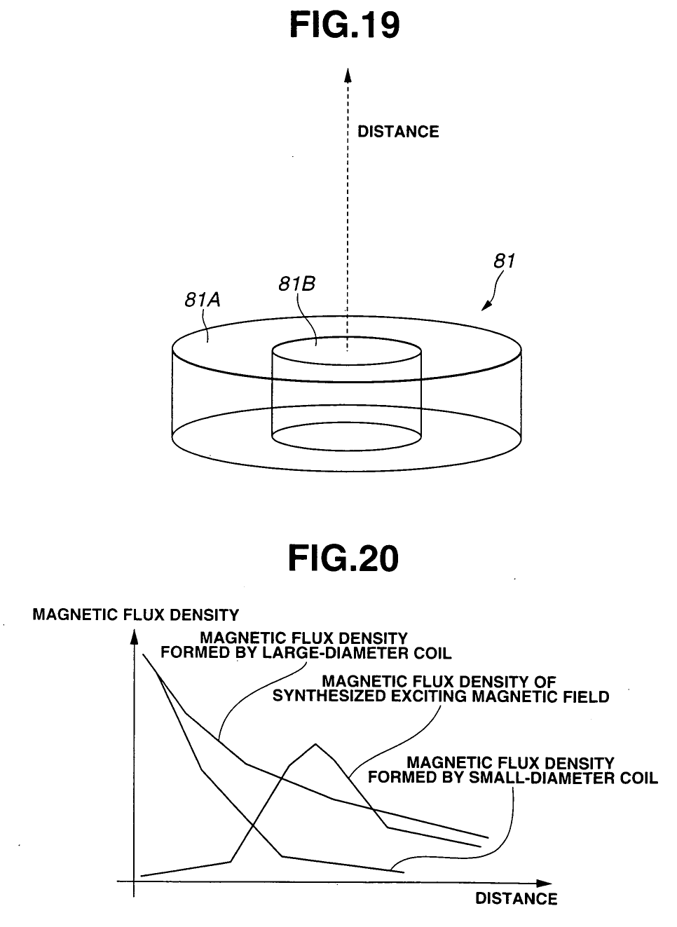

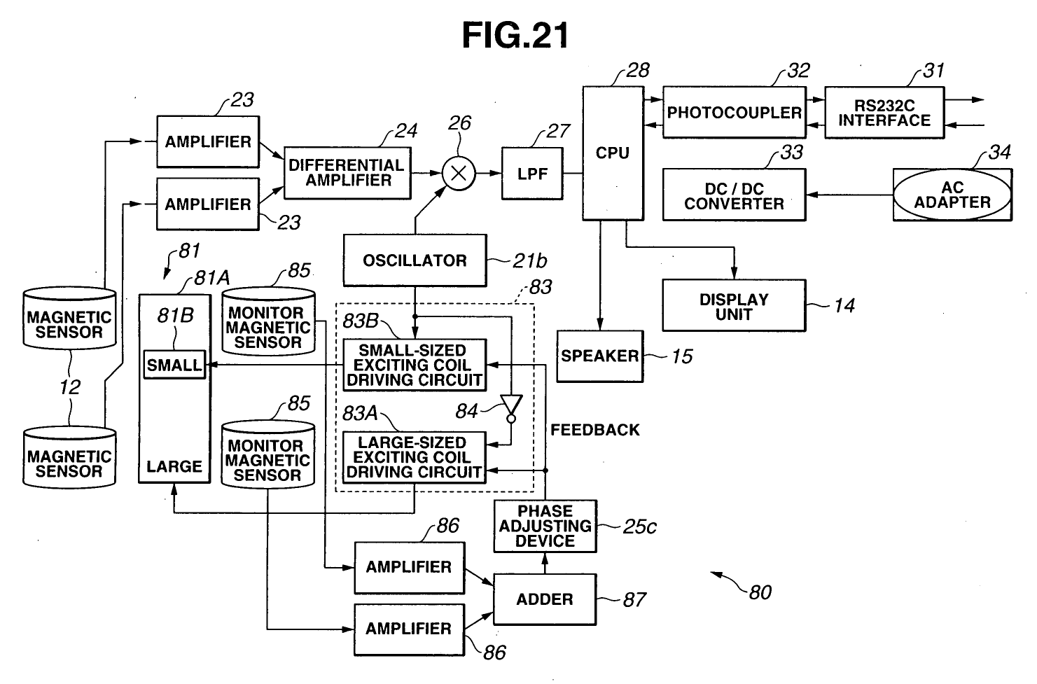

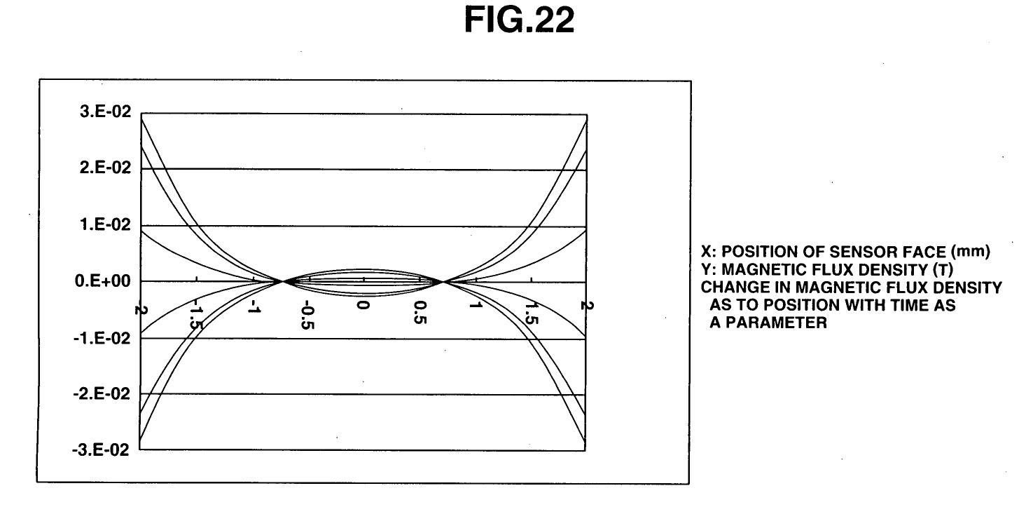

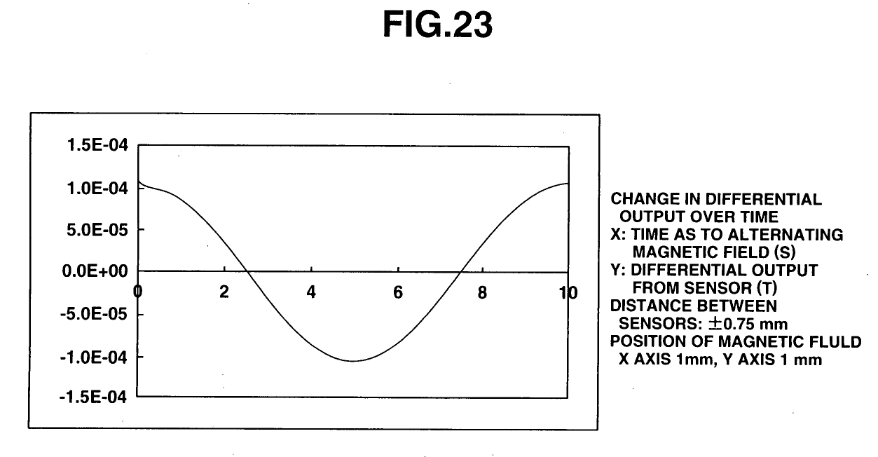

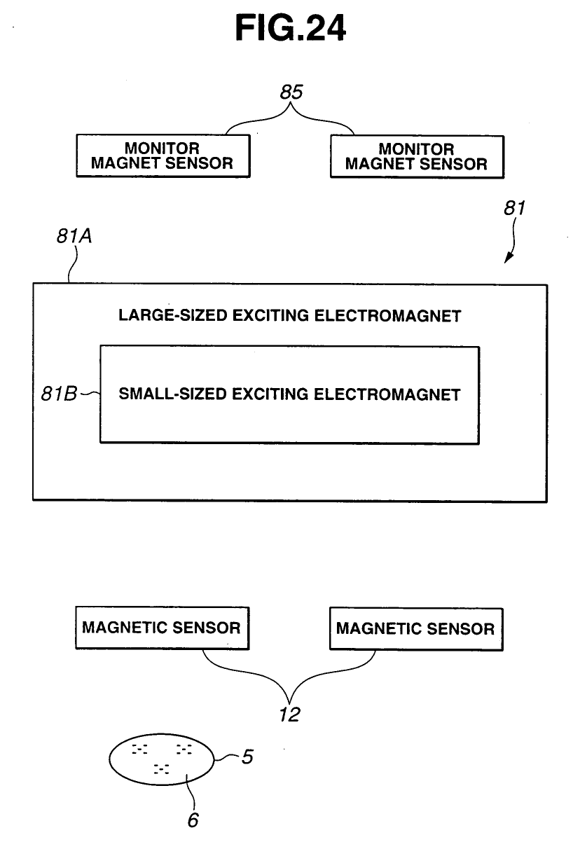

FIGS. 18 through 24 relate to a third embodiment according to the present invention, wherein FIG. 18 is an explanatory diagram which illustrates a probe forming a magnetic fluid detecting apparatus according to the third embodiment, FIG. 19 is an explanatory diagram which illustrates exciting electromagnets shown in FIG. 18, FIG. 20 is a chart which shows the magnetic flux density of the exciting magnetic field formed by the exciting electromagnets shown in FIG. 19, FIG. 21 is a circuit block diagram which shows a circuit configuration of the magnetic fluid detecting apparatus according to the third embodiment, FIG. 22 is a chart which shows the change in the magnetic flux density over time, formed by the exciting electromagnets AC-driven by an exciting coil driving circuit shown in FIG. 20, FIG. 23 is a chart which shows the differential output detected by the magnetic sensors in a case that there is magnetic fluid, and FIG. 24 is an explanatory diagram for describing an arrangement including monitor magnetic sensors.

While description has been made regarding an arrangement including one exciting electromagnet in the above second embodiment, an arrangement according to the third embodiment includes two exciting electromagnets. The configuration other than the two exciting electromagnets is generally the same as with the above-described second embodiment, so description thereof will be omitted, and description will be made regarding the same components with the same reference numerals.

As shown in FIG. 18, a magnetic fluid detecting apparatus 80 according to the third embodiment comprises a probe 82 having a configuration wherein an exciting electromagnet 81 formed of a combination of two exciting electromagnets combined with reverse polarities is included therein on the tip side thereof. That is to say, the exciting electromagnet 81 according to the present embodiment includes a large-sized electromagnet 81A with a large diameter on the perimeter thereof and a small-sized electromagnet 81B with a small diameter in the core of the large-sized electromagnet, with reverse polarities, as shown in FIG. 19.

With regard to the large-sized exciting electromagnet 81A, the greater the diameter thereof is, the farther the magnetic flux density formed by the large-diameter coil thereof reaches. On the other hand, the small-sized exciting electromagnet 81B has a small diameter, and accordingly, the magnetic flux density is formed by the small-diameter coil thereof generally with the same magnitude as with the large-sized exciting electromagnet 81A in the range up to a predetermined distance, but with a magnitude being reduced just beyond the predetermined distance. Furthermore, with the exciting electromagnet 81 according to the present embodiment, the magnetic flux densities formed by the large-diameter coil and the small-diameter coil have polarities reverse one another, cancel out each other, and are synthesized as shown in FIG. 20. Note that FIG. 20 shows the magnetic flux density as to the distance on the longitudinal axis of the exciting magnet 81.

As shown in FIG. 20, with regard to the synthesized magnetic flux density of the exciting magnetic field, the farther from the exciting electromagnet in a range up to a position of which distance is determined by the size of the diameter, the greater the magnetic flux density is, and on the other hand, the farther from the exciting electromagnet beyond this position, the smaller the magnetic flux density is.

Next, description will be made regarding a circuit configuration of the magnetic fluid detecting apparatus 80 according to the third embodiment with reference to FIG. 21.

The magnetic fluid detecting apparatus 80 comprises an exciting coil driving circuit 83 for driving the exciting electromagnet 81 with the oscillating frequency from the oscillator 21b based upon the phase adjusted by the phase adjusting device 25c. The exciting coil driving circuit 83 comprises a small-sized exciting coil driving circuit 83B for driving the small-sized exciting electromagnet 81B and a large-sized coil driving circuit 83A for driving the large-sized exciting electromagnet 81A.

Note that with the large-sized coil driving circuit 83A, the phase of the signals from the oscillator 21b is inverted by an inverting amplifier 84.

Here, FIG. 22 shows an example of the change in the magnetic flux density over time formed by the exciting magnet 81 AC-driven by the exciting coil driving circuit 83. Note that FIG. 22 shows the magnetic flux density as to the horizontal distance from the center axis of the exciting electromagnet 81. The magnetic flux densities formed by the large-diameter coil and the small-diameter coil cancel out each other as described above, and the magnetic density formed by the exciting electromagnet 81 always have two constant zero points independent of time.

Accordingly, with the present embodiment, the magnetic sensors 12 are disposed at positions matching the above-described two zero points, and thus, the magnetic sensors 12 are not influenced by the change in the magnetic field over time (AC driving components) (noise) due to the exciting magnet 81.

Thus, the magnetic sensors 12 can detect only the local magnetic field from the magnetic fluid 6 in the event that there is magnetic fluid 6, that is to say, the differential output between these outputs exhibits only the local magnetic components as shown in FIG. 23.

Furthermore, the magnetic fluid detecting apparatus 80 includes two monitor magnetic sensors 85 for monitoring the magnetic field due to the exciting electromagnet 81 as shown in FIG. 24. The outputs from the monitor magnetic sensors 85 are each amplified by amplifiers 86 for monitors, and are input to an adder 87. The adder 87 outputs to the phase adjusting device 25c an integral value obtained by adding the outputs from the monitor magnet sensors 85.

The phase adjusting device 25c performs feedback wherein the amplitude of the driving current applied to the exciting electromagnet 81 is adjusted corresponding to the integral value from the adder 87 so as to fix the magnetic field formed by the exciting electromagnet 81. Thus, the magnetic fluid detecting apparatus 80 can adjust the magnetic field formed by the exciting electromagnet 81 so that the magnetic fluid detecting apparatus 80 is not influenced by the magnetic field (noise) formed by the exciting electromagnet 81 due to the winding method for the exciting coil 54 or the difference in the layout.

Note that the circuit configuration other than the above-described configuration is the same as with the above-described first embodiment.

The magnetic fluid detecting apparatus 80 having such a configuration is used for identifying the sentinel lymph node 5 where the magnetic fluid 6 has been accumulated in the same way as with the above-described first embodiment.

The magnetic fluid detecting apparatus 80 excites a space near the probe using the exciting electromagnet 51, and measures the spatial gradient of the magnetic field distribution (magnetic flux density) using the magnetic sensors 12.

At this time, the monitor magnetic sensors 85 detect the magnetic field formed by the exciting electromagnet 81. The control device 4 performs feedback wherein the amplitude of the driving current applied to the exciting electromagnet 81 is adjusted so as to fix the magnetic field formed by the exciting electromagnet 81 so that the magnetic sensors 12 are not influenced by the magnetic field (noise) formed by the exciting electromagnet 81.

Thus, the magnetic fluid detecting apparatus 80 according to the third embodiment can more efficiently identify sentinel lymph node 5 in a surer manner, as well as having the same advantages as with the above-described second embodiment.

(Fourth Embodiment)

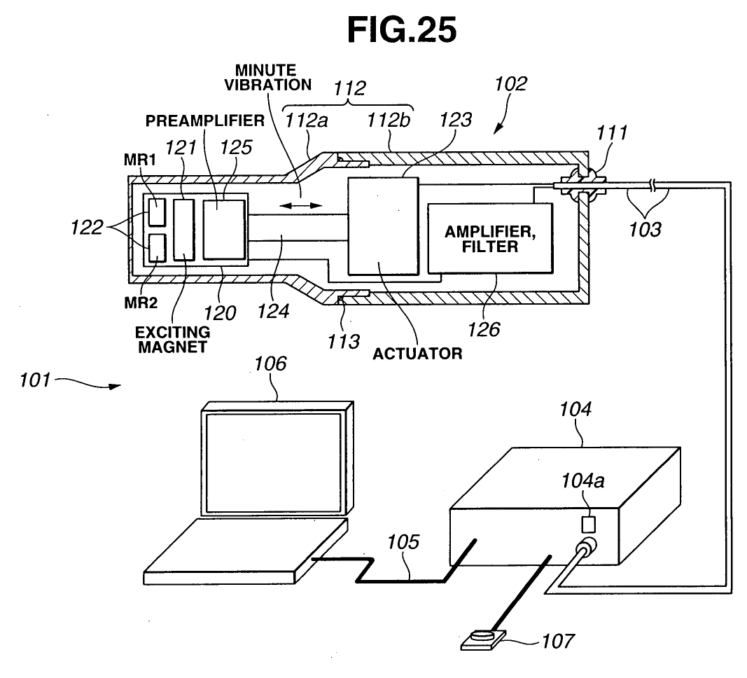

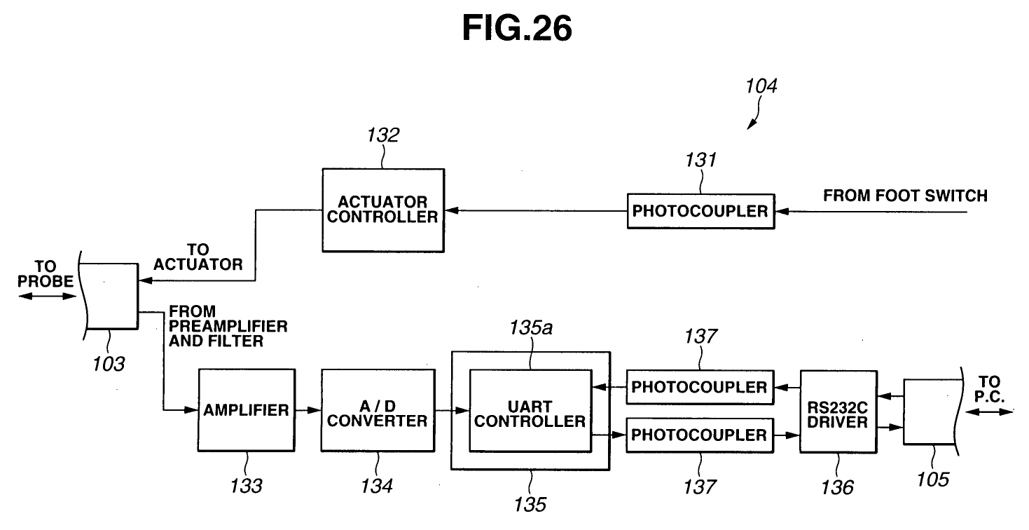

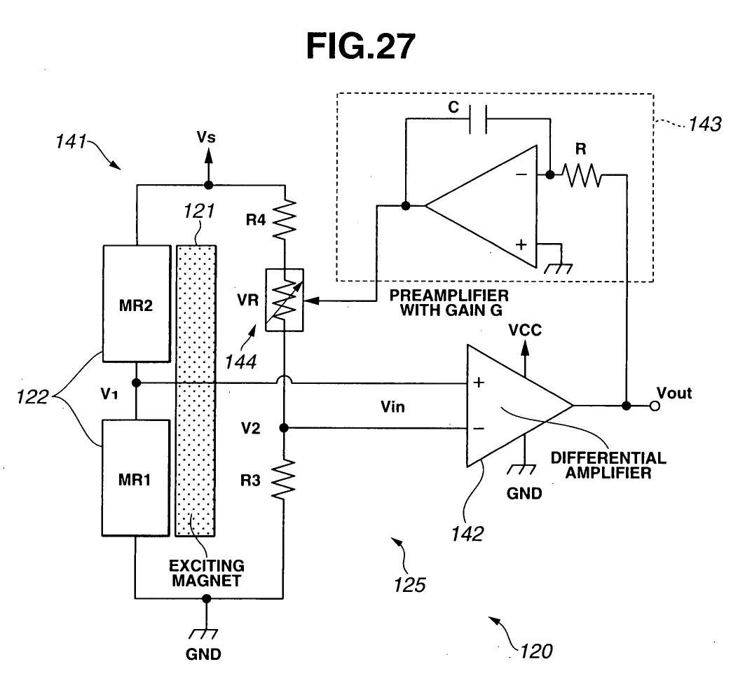

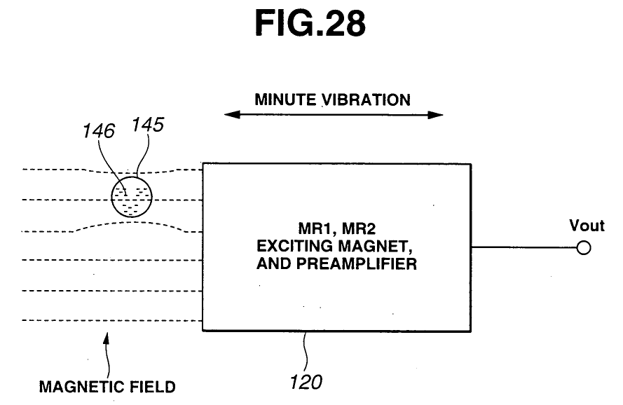

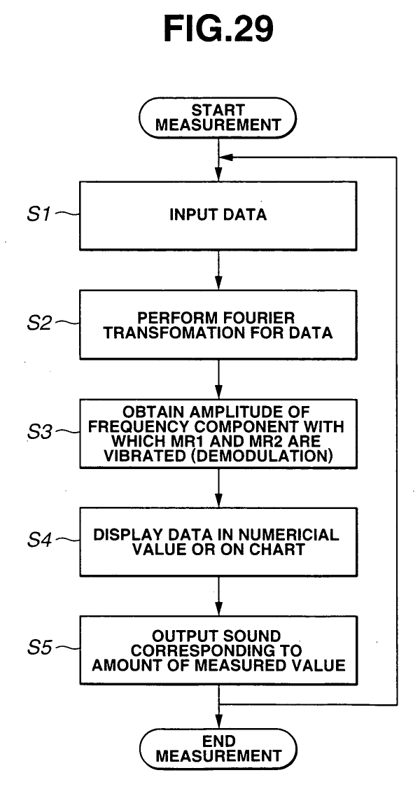

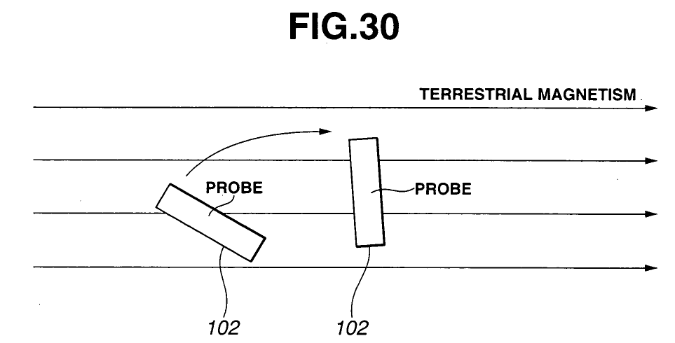

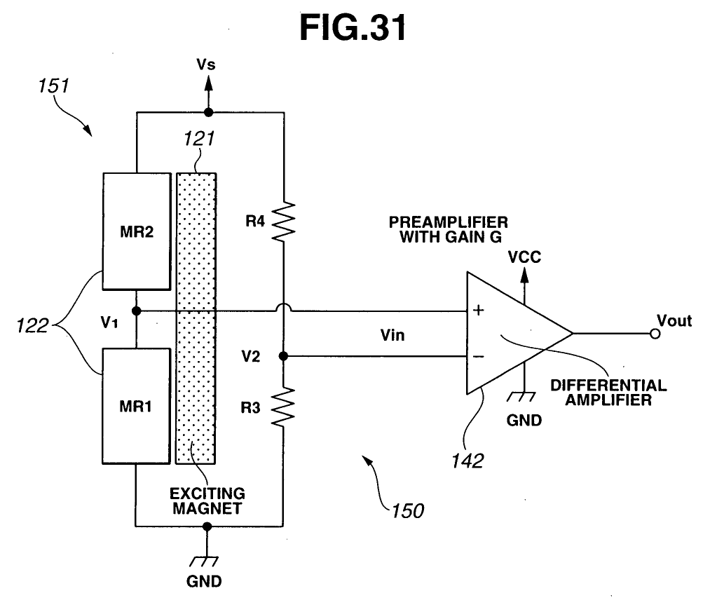

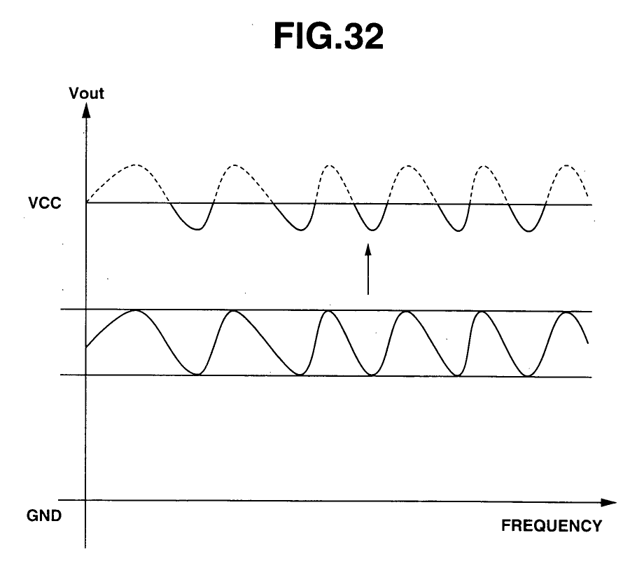

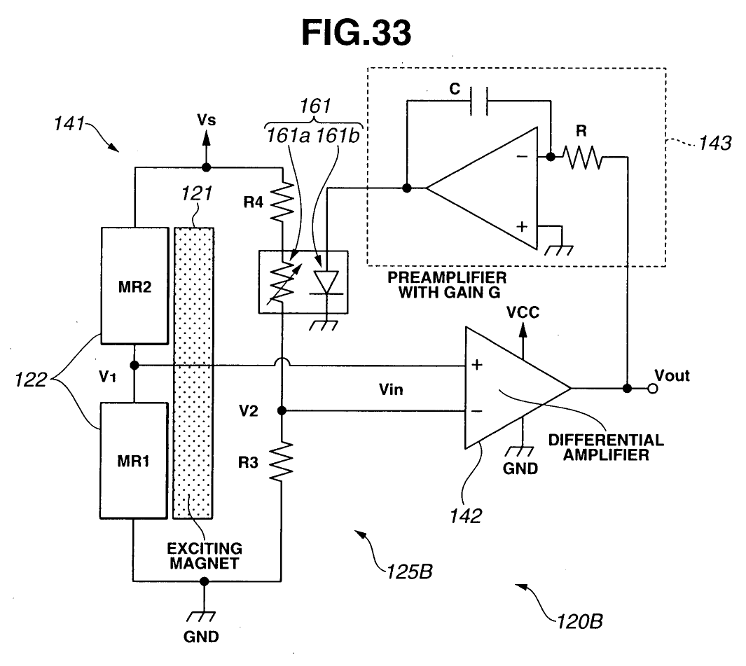

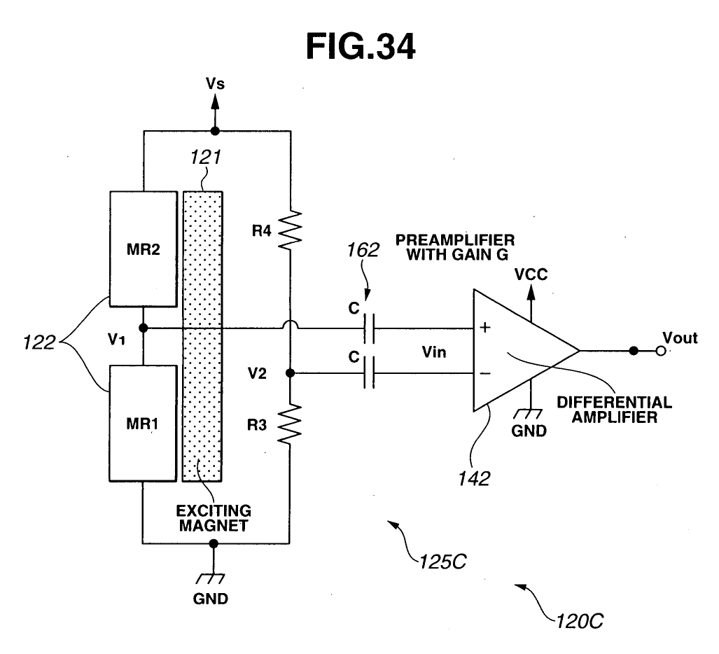

FIG. 25 through FIG. 34 relate to a fourth embodiment according to the present invention, wherein FIG. 25 is an overall configuration diagram which illustrates a magnetic fluid detecting apparatus for identifying sentinel lymph nodes according to a first embodiment of the present invention, FIG. 26 is a circuit block diagram which shows a circuit configuration of a control device, FIG. 27 is a circuit block diagram which shows a circuit configuration of a sensor unit according to the present embodiment, FIG. 28 is a schematic diagram which illustrates the sensor unit detecting magnetic fluid accumulated in a sentinel lymph node, FIG. 29 is a flowchart which shows the control performed by a P.C., FIG. 30 is a schematic diagram which illustrates the probe with the attitude changed in the terrestrial magnetism, FIG. 31 is a circuit block diagram of the sensor unit having an assumed circuit configuration, FIG. 32 is a chart which shows the output Vout from the sensor unit shown in FIG. 31, FIG. 33 is a circuit block diagram which shows a first modification of the sensor unit, and FIG. 34 is a circuit block diagram which shows a second modification of the sensor unit.

As shown in FIG. 25, a magnetic fluid detecting apparatus 101 according to the fourth embodiment of the present invention principally comprises a probe 102 including an exciting magnet and magnetic sensors described later therein on the tip side thereof, which can be inserted in the body cavity, and a control device 104, which is connected to the probe 102 through a connecting cable 103, for controlling the probe 2.

The control device 104 is connected to a personal computer (which will be referred to as “P.C.” hereafter) 106 through an RS232C cable 105 so as to be controlled by the P.C. 106. Furthermore, the control device 104 is connected to a foot switch 107. Upon the foot switch 107 being turned on, the control device 104 supplies electric power to the probe 102.

Note that reference numeral 104a denotes a power switch provided on the front panel of the control device 104. Furthermore, an arrangement may be made wherein an unshown hand switch is connected to the control device 104 instead of the foot switch 107. The hand switch may be water-proof or disposable. Furthermore, an arrangement may be made wherein the hand switch can be mounted to the probe 102, or an arrangement may be made wherein the hand switch can be used independently.

The probe 102 has the connecting cable 103 extending therefrom through a water-proof grommet 111, and is formed watertight with a water-proof casing member 112. The casing member 112 comprises two members of a tip-side casing member 112a and a rear-side casing member 112b, and an O-ring 113 is provided therebetween. Note that the tip-side casing member 112a is formed of a non-magnetic material so as to prevent the exciting magnet and the magnetic sensors included on the tip side from being magnetically influenced.

The probe 102 has a configuration wherein the sensor unit 120 includes an exciting magnet 121 for exciting magnetic fluid accumulated in sentinel lymph nodes within the subject across a space near the probe, and MR sensors (magnetic resistance sensors) 122 serving as magnetic sensors for detecting the distortion of the local magnetic field distribution (the change in the magnetic flux density) due to the excited magnetic fluid.

Note that with the present embodiment, two MR sensors 122 of MR 1 and MR 2 form a four-terminal bridge as described later.

An actuator 123 included on the probe-base side performs minute vibration of the sensor unit 120 in the longitudinal direction. Note that an actuator controller of the control device 104 described later performs on/off control of the actuator 123.

Accordingly, the magnetic fluid detecting apparatus 101 modulates the local magnetic field due to the magnetic fluid excited by the exciting magnet 121, detects the distortion of the local magnetic field distribution (the change in the magnetic flux density) using the MR sensors 122 (sensors MR 1 and MR 2), obtains the difference between the outputs from the two MR sensors 122 (sensors MR 1 and MR 2), and furthermore performs demodulation, thereby removing the magnetic noise with frequencies other than modulation frequency from the terrestrial magnetism or other electric devices. Note that the sensor unit 120 is connected to the actuator 123 through a joint 124 formed of non-magnetic material. Note that the MR sensors 122 (sensors MR 1 and MR2) measure the change in the resistance due to the magnetic field.

Accordingly, the sensor unit 120 according to the present embodiment further includes a preamplifier 125 for amplifying the outputs from the MR sensors 122 (sensors MR 1 and MR 2), as well as performing minute vibration thereof in the longitudinal direction. Accordingly, the magnetic fluid detecting apparatus 101 prevents a problem that the MR sensors 122 (sensors MR 1 and MR 2) cannot measure the precise change in the magnetic field due to the change in the resistance of the lead wires between the MR sensors 122 (sensors MR 1 and MR 2) and the preamplifier 125 due to the change in the shape of the lead wires therebetween.

Furthermore, the probe 102 includes a circuit board 126, having an amplifier for amplifying the signals output from the sensor unit 120 to the control device 104, a filter for performing noise removal, and the like, on the base side thereof.

First, description will be made regarding the internal configuration of the control device 104.

As shown in FIG. 26, the control device 104 includes an actuator controller 132, formed of relays or the like, for example, for performing on/off control for the actuator 123 of the probe 102 based upon the on/off signals output from the foot switch 107 through a photocoupler 131. Note that the photocoupler 131 is employed for electrically insulating the on/off signals from the foot switch 107.

Furthermore, the control device 104 includes an amplifier 133 for amplifying the signals from the sensor unit 120 of the probe 102, an A/D converter 134 for performing A/D converting for the signals amplified by the amplifier 133, and a CPU (Central Processing Unit) 135 for performing signal processing for the digital signals subjected to conversion by the A/D converter 134.

Note that the CPU 135 includes a UART (Universal Asynchronous Receiver Transmitter) controller 135a for converting digital signals output from the A/D converter 134 in parallel into serial output signals.

Furthermore, the control device 104 includes an RS232C driver 136 for performing communication between the CPU 135 and the P.C. 106 through the RS232C cable 105., and the transmission/reception of the signals between the RS232C driver 136 and the CPU 135 is electrically insulated with the photocoupler 137.

Accordingly, the control device 104 detects magnetic fluid accumulated within the subject, while communicating with the P.C. 106, based upon the control performed by the P.C. 106 operated by the surgeon. Note that the P.C. 106 notifies the surgeon of the presence or absence of the detected magnetic fluid based upon the flowchart described later.

Next, description will be made regarding the circuit configuration of the sensor unit 120 according to the present embodiment with reference to FIG. 27.

As shown in FIG. 27, the sensor unit 120 according to the present embodiment includes a four-terminal bridge 141 formed of a combination of two MR sensors 122 (sensors MR 1 and MR 2) and fixed resistors R3 and R4. The preamplifier 125 has a configuration wherein the outputs Vin (V1, V2) from the four-terminal bridge 141 are subjected to differential amplification by a differential amplifier 142, whereby Vout is output.

An arrangement according to the present embodiment includes a feedback circuit 143 for performing feedback for the output Vout from the preamplifier 125 for removing offset. Furthermore, the arrangement includes a condenser C, whereby the output from the feedback circuit 1043 is held in the event that the output Vout is zero.

Furthermore, the sensor unit 120 includes a variable resistor VR 144 on the R4 side of the four-terminal bridge 141, serving as a resistor with a value being changed corresponding to the output from the feedback circuit 143, thereby forming offset variable means for offsetting the difference between the outputs Vin (V1, V2) from the MR sensor 122 (sensors MR 1 and MR2).

Note that the output Vout from the differential amplifier 142 with regard to the input Vin (output from the four-terminal bridge 41) is represented by Expression (1).

$$\begin{matrix}Vout = G Vin \\ = G ( V1 - V2 ) \\ = \frac{G Vs ( MR1 ( R4 + VR ) - MR2 R3 )}{( MR1 + MR2 ) ( R3 + ( R4 + VR ) )}\end{matrix} \qquad ( 1 )$$

Here, G denotes the gain of the preamplifier 125, Vs denotes the standard voltage value, VR denotes variable resistance value, MR 1 denotes the resistance value of the sensor MR 1, and MR 2 denotes the resistance value of the sensor MR 2.

As can be understood from Expression (1), in order to make the output Vout zero,

MR1(R4+VR)=MR2 R3

needs to be satisfied.

Accordingly, in the event that the variable resistance of the variable resistor VR 144 is adjusted to the value shown in the following Expression (2), the output Vout becomes zero.

VR=MR2 R3(MR1)−R4 (2)

That is to say, with the present embodiment, the variable resistance of the variable resistor VR 144 is adjusted corresponding to the output from the preamplifier 125 based upon the Expression (2), thereby offsetting the difference between the outputs Vin (V1, V2) from the MR sensors 122 (sensors MR1 and MR2).

Note that the offset processing requires time corresponding to the time constant determined by the condenser C and the resistor R, and accordingly, the change in signals due to the magnetic fluid is not removed. Furthermore, an arrangement may be made wherein a variable resistance with the resistance value being changed by an unshown motor is employed for the variable resistor VR 144.

The magnetic fluid detecting apparatus 101 having such a configuration is used for identifying sentinel lymph nodes by detecting the magnetic fluid accumulated in the sentinel lymph nodes within the subject.

First, the surgeon inserts an unshown puncture needle into the lower layer of the affected portion of the subject in order to inject magnetic fluid near the affected portion. The magnetic fluid locally injected near the affected portion moves to lymph vessels from the injected portion, reaches sentinel lymph nodes after five to 15 minutes, and is accumulated in the sentinel lymph nodes.

Subsequently, the surgeon surgically inserts the probe 102 of the magnetic fluid detecting apparatus 101 into the body cavity through an unshown trocar. The surgeon detects the magnetic fluid accumulated in the sentinel lymph nodes while moving the tip of the probe 102 as to the portion near the affected portion of the patient.

At this time, the surgeon presses down the foot switch 107 so as to be continuously on only in the event of performing detection (measurement) for the magnetic fluid near the affected portion of the patient.

As a result, with the control device 104, the on signal is transmitted from the foot switch 107 to the actuator controller 132, the electric power is supplied from the actuator controller 132 to the actuator 123 so as to drive the actuator 123, whereby minute vibration is performed with a predetermined oscillating frequency. Accordingly, with the probe 102, minute vibration of the sensor unit 120 is performed with a predetermined oscillating frequency, and more specifically, minute vibration of the exciting magnet 121 and the MR sensors 122 is performed in the direction orthogonal to the direction of the line including the two MR sensors, and in the longitudinal direction including the magnet 121.

With the probe 102, the exciting magnet 121 excites a space near the probe, and the MR sensors 122 measure the spatial gradient of the magnetic field distribution (magnetic flux density).

In the event that there is a sentinel lymph node 145 near the affected portion of the patient as a subject, magnetic fluid 146 is accumulated in the sentinel lymph node 145, and accordingly, local distortion of the magnetic field distribution occurs due to absorption of the magnetic field applied by the exciting magnet 121 around the magnetic fluid 146, whereby the change in the spatial distribution of the magnetic field distribution (magnetic flux density) occurs. With the probe 102, minute vibration of the exciting magnet 121 and the MR sensors 122 is performed with a predetermined oscillating frequency in the longitudinal direction, whereby modulation is performed for the local magnetic field due to the magnetic fluid 146 excited by the exciting magnet 121. As a result, the distortion of the local magnetic field distribution (the change in the magnetic flux density) due to the magnetic fluid 146, subjected to modulation, is detected by the MR sensors 122 (sensors MR1 and MR2).

The difference between the outputs from the MR sensors 122 (sensors MR1 and MR2) is obtained and amplified by the differential amplifier 142, is amplified by an amplifier on the circuit board 126, is subjected to filtering processing by a filter, and is output to the control device 104.

Subsequently, with the control device 104, the signals from the sensor unit 120 of the probe 102 are amplified by the amplifier 133, are subjected to A/D conversion by the A/D converter 134, are converted into serial output signals by the UART controller 135a of the CPU 135, and the serial output signals are transmitted to the RS232C driver 136, and are output to the P.C. 106 through the RS232C cable 105.

Here, P.C. 106 performs control for notifying the surgery based upon the flowchart shown in FIG. 29.

As shown in FIG. 29, the signals from the control device 104 are input to the P.C. 106 as data (Step S1). Subsequently, the P.C. 106 performs the known Fourier transformation for the input data (Step S2).

Next, the P.C. 106 obtains the amplitude of the frequency component (oscillating frequency) wherein the MR sensors 122 (sensors MR1 and MR2) are vibrated, and performs demodulation (Step S3).

Subsequently, the P.C. 106 converts the demodulated data into numerical values or a graph, representing the change in the magnetic flux density, so as to be displayed on a monitor (Step S4), or output sound with an accessory speaker corresponding to the magnitude of the measured values so as to notify the surgeon (Step S5).

The P.C. 106 consecutively performs processing shown in the above-described Steps S1 through S5 until the surgeon releases the foot switch 107 so that measurement using the probe 102 ends.

With the magnetic fluid detecting apparatus 101, the magnitude of the spatial distortion of the magnetic field due to the magnetic fluid 146 is insufficient during measurement, and accordingly, there is the need to improve the sensitivity by increasing the gain of the preamplifier. 125. At this time, the surgeon might change the attitude of the probe 102 in the terrestrial magnetism as shown in FIG. 30, for example.

Making an assumption that the sensor unit includes a preamplifier 150 having a configuration wherein the difference between the outputs Vin (V1, V2) from a four-terminal bridge 151 as shown in FIG. 31 is obtained and amplified by the differential amplifier 142, whereby Vout is output, the output Vout from the MR sensors 122 (sensors MR1 and MR2) is changed due to the influence of the terrestrial magnetism or the like, leading to a problem that saturation of the output from the preamplifier 150 occurs (i.e., the output Vout exceeds the power voltage Vcc) as shown in FIG. 32.

Note that FIG. 31 is a circuit block diagram of the sensor unit in the event of making an assumption that the sensor unit has the above-described circuit configuration, and FIG. 32 is a chart which shows the output Vout from the sensor unit shown in FIG. 31.

In this case, detection of the magnetic fluid 146 becomes difficult, which might lead to a problem that the accurate position of the sentinel lymph node 45 cannot be identified.

Note that the output Vout from the differential amplifier 142 shown in FIG. 31 is represented by the following Expression (3) with regard to the input (output from the four-terminal bridge 141) Vin.

$$\begin{matrix}Vout = G Vin \\ = G ( V1 - V2 ) \\ = \frac{G Vs ( MR1 R4 - MR2 R3 )}{( MR1 + MR2 ) ( R3 + R4 )}\end{matrix} \qquad ( 3 )$$

Here, G denotes the gain of the preamplifier 25, Vs denotes the standard voltage value, MR1 denotes the resistance value of the sensor MR1, and MR2 denotes the resistance value of the sensor MR2.

The present embodiment has the above-described configuration wherein the variable resistance value of the variable resistor VR 144 can be changed corresponding to the output from the preamplifier 125 based upon the Expression (2), and thus the difference between the outputs Vin (V1, V2) from the MR sensors 122 (sensors MR1 and MR2) can be offset.

Thus, with the magnetic fluid detecting apparatus 101 according to the present embodiment, saturation of the output from the preamplifier 125 does not occur (the output from the preamplifier 125 does not exceed the power voltage Vcc), and thus the magnetic fluid 146 can be detected. Thus, the magnetic fluid detecting apparatus 101 according to the present embodiment can identify the precise position of the sentinel lymph node 145.

Note that the sensor unit may have a configuration including a CdS photocoupler as shown in FIG. 33.

FIG. 33 is a circuit block diagram which shows a first modification of the sensor unit.

As shown in FIG. 33, a sensor unit 120B has a configuration having a preamplifier 125B including a CdS photocoupler 161.

The CdS photocoupler 161 is a photocoupler formed of a combination of a CdS 161a which is a device exhibiting the photo-conductive effect wherein the resistance (electric conductivity) is changed due to the intensity of the light (energy greater than the forbidden band width), and an LED (Light Emitting Diode) 161b. With the CdS photocoupler 161, the LED 161b emits light, the CdS 161a receives the emitted light, and accordingly, the resistance value thereof changes, and thus, the difference between the outputs Vin (V1, V2) from the MR sensors 122 (sensors MR1 and MR2) can be offset.

As described above, the sensor unit 120B includes the CdS photocoupler 161 instead of the variable resistor VR 144, and thus, the sensor unit 120B can eliminate a problem of non-linearity due to a normal photocoupler formed of normal transistors or FETs.

Furthermore, in a case of employing high modulation frequency, the sensor unit may have a configuration wherein the MR sensors 122 (sensors MR1 and MR2) and the preamplifier 125 forms an AC coupling configuration, as shown in FIG. 34.

FIG. 34 is a circuit block diagram which shows a second modification of the sensor unit.

As shown in FIG. 34, the sensor unit 120C includes a preamplifier 125C having an AC coupling configuration formed of the MR sensors 122 (sensors MR1 and MR2) and the preamplifier 125 using condensers 162 for cutting off the DC component of the outputs Vin (V1, V2) from the MR sensors 122 (sensors MR1 and MR2), without using a variable resistor.

As described above, the sensor unit 120C has the AC coupling configuration formed of the MR sensors 122 (sensors MR1 and MR2) and the preamplifier 125, thereby having a simple configuration without using the variable resistor VR 144.

(Fifth Embodiment)

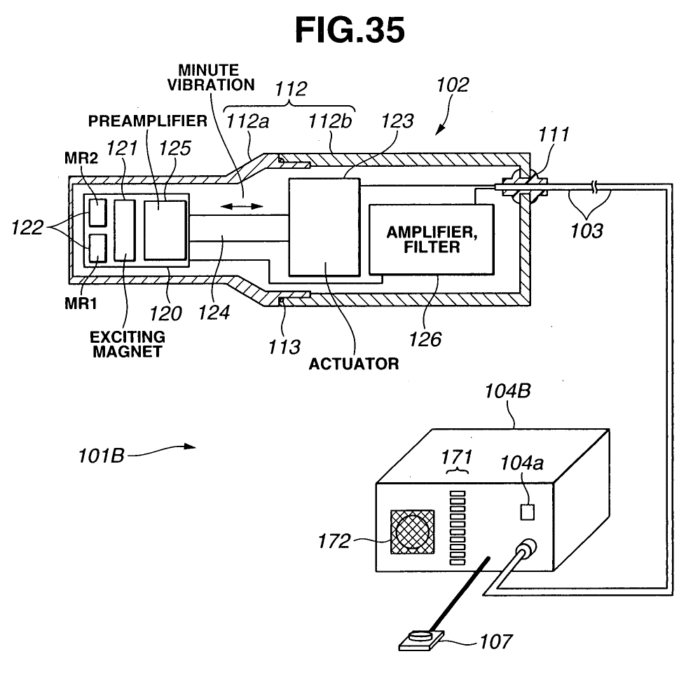

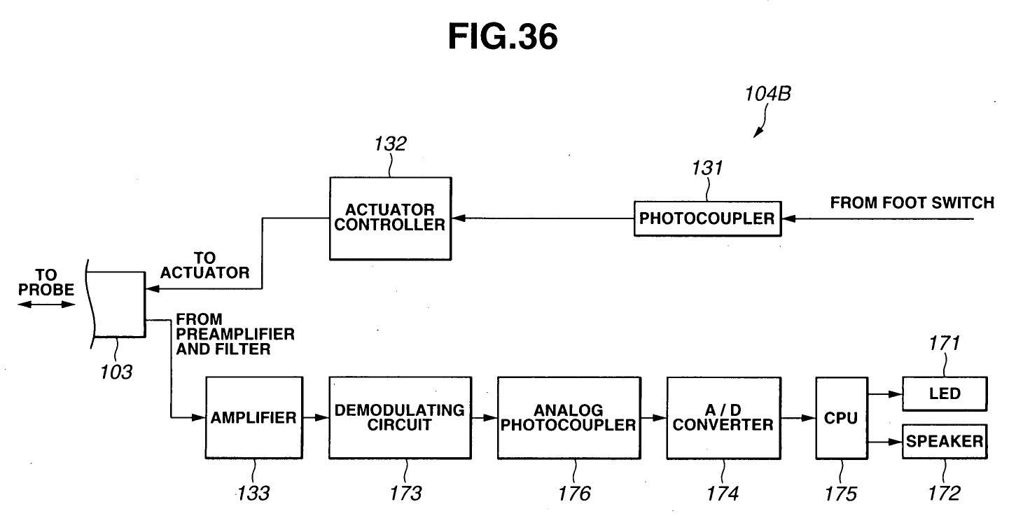

FIGS. 35 and 36 relate to a fifth embodiment according to the present invention, wherein FIG. 35 is an overall configuration diagram which illustrates a magnetic fluid detecting apparatus according to the fifth embodiment of the present invention, and FIG. 36 is a circuit block diagram which shows the circuit configuration of a control device shown in FIG. 35.

The fifth embodiment has a configuration including no P.C., only a probe and a control device. The configurations other than the configuration is the same as with the above-described fourth embodiment, so description thereof will be omitted, and description will be made denoting the same components with the same reference numerals.

That is to say, a magnetic fluid detecting apparatus 101B according to the fifth embodiment has a configuration including only the same probe 102 as in the above-described fourth embodiment and a control device 104B without using a P.C., as shown in FIG. 35.

The control device 104B includes a display unit 171 formed of LEDs or the like for displaying the change in the magnetic flux density detected by the MR sensors 122 (sensors MR1 and MR2), and a speaker 172 for giving notice of the change in the magnetic flux density by audio, on the front panel thereof.

Note that, with the present embodiment, while FIG. 35 shows an arrangement wherein the display unit 171 comprises a indicator formed of LEDs or the like, an arrangement may be made wherein image display is performed using an LCD (Liquid Crystal Display) as the display unit 171.

Next, description will be made regarding the configuration of the control device 104B with reference to FIG. 36.

FIG. 36 is a circuit block diagram which shows the circuit configuration of the control device 104.

As shown in FIG. 36, the control device 104B comprises a demodulating circuit 173 for demodulating the signals from the amplifier 133 into analog signals, an A/D converter 174 for performing A/D conversion for the analog signals demodulated by the demodulating circuit 173, and a CPU 175 for driving the LEDs of the display unit 171 or the speaker 172 according to the digital signals converted by the A/D converter 174 so as to perform the same notifying processing as with the P.C. Note that the demodulating circuit 173 is connected to the A/D converter 174 through an analog photocoupler 176 for electric insulating.

The magnetic fluid detecting apparatus 101B having such a configuration is used for detecting the magnetic fluid 146 accumulated in the sentinel lymph node 145 within the subject so as to identify the sentinel lymph node 145 in the same way as in the above-described fourth embodiment.

With the control device 104B, the signals from the sensor unit 120 of the probe 102 are amplified by the amplifier 133, following which the amplitude of the frequency component (oscillating frequency) with which the MR sensors 122 (sensors MR1 and MR 2) are vibrated, is obtained and demodulated, and the LEDs of the display unit 171 are turned on, or sound is generated from the speaker 172 so as to giving notice, based upon the demodulated data as the change in the magnetic flux density.

Note that the operations other than in the above description are the same as with in the above-described fourth embodiment, so description thereof will be omitted.

As a result, the magnetic fluid detecting apparatus 101B according to the present fifth embodiment has a simple configuration without using a P.C., as well as having the same advantages as in the above-described fourth embodiment.

(Sixth Embodiment)

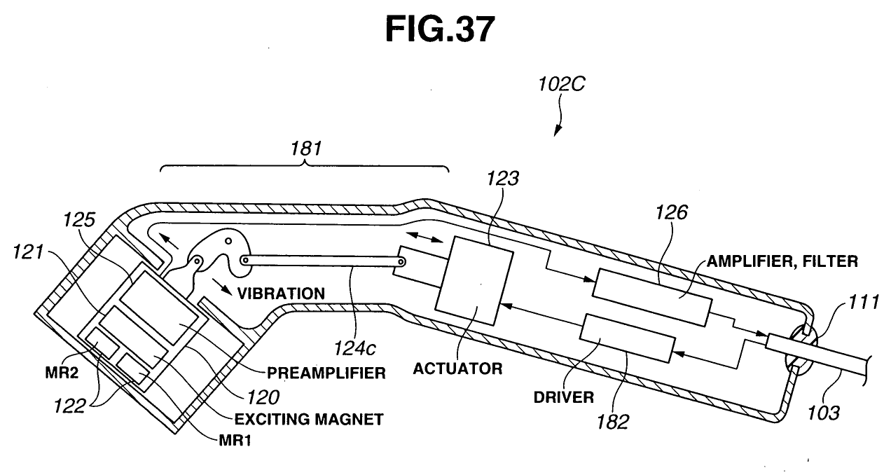

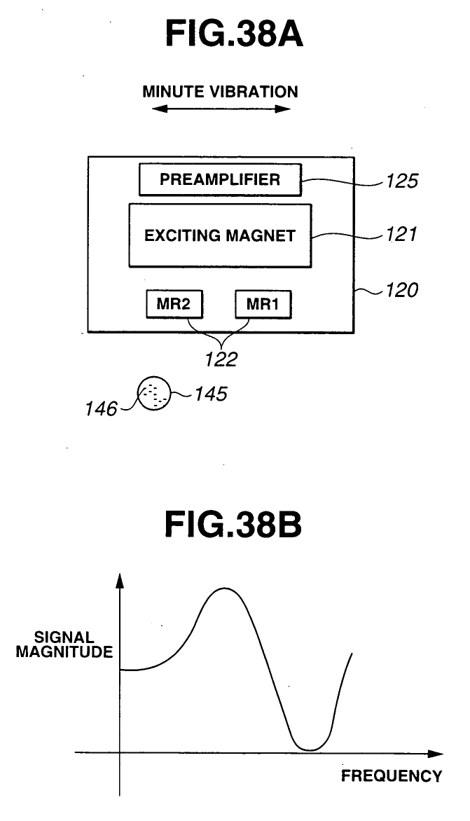

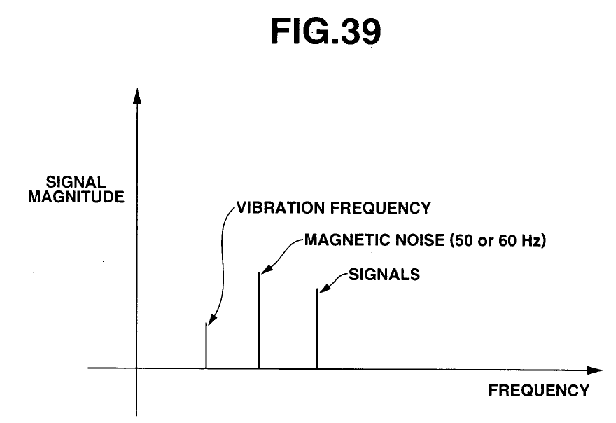

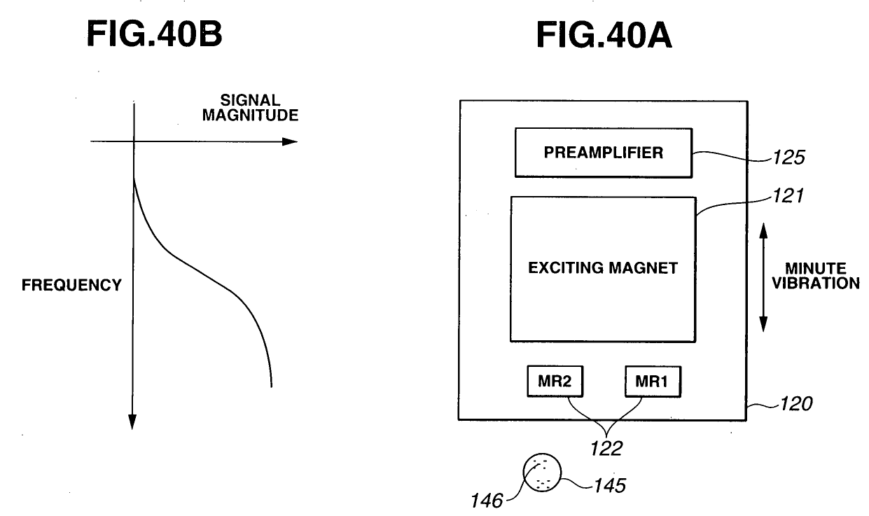

FIGS. 37 through 40A relate to a sixth embodiment according to the present invention, wherein FIG. 37 is an explanatory diagram which illustrates a probe forming a magnetic fluid detecting apparatus according to the sixth embodiment of the present invention, FIG. 38A is a schematic diagram which illustrates minute vibration of a sensor unit being performed in the direction parallel to the line where two MR sensors have been disposed, FIG. 38B is a chart which shows signals from the MR sensors of the sensor unit shown in FIG. 38A, FIG. 39 is a chart which shows signals obtained from the sensor unit shown in FIG. 38A, which have been subjected to the Fourier transformation, FIG. 40A is a schematic diagram which illustrates minute vibration of the sensor unit being performed in the direction orthogonal to the line where the two MR sensors have been disposed, and in the longitudinal direction including the magnet, and FIG. 40B is a chart which shows signals from the MR sensors of the sensor unit shown in FIG. 40A.

While description has been made regarding the arrangements according to the above fourth and fifth embodiments, wherein minute vibration of the sensor unit is performed in the direction orthogonal to the line where the two MR sensors have been disposed, and in the longitudinal direction thereof including the magnet, the present sixth embodiment has a configuration wherein minute vibration of the sensor unit is performed in the direction parallel to the line where the two MR sensors have been disposed. The other configuration is generally the same as with the above-described fourth and fifth embodiments, so description thereof will be omitted, and description will be made regarding the same components with the same reference numerals.

That is to say, the magnetic fluid detecting apparatus according to the present sixth embodiment includes a probe 102C having a configuration wherein minute vibration of the sensor unit 120 is performed in the direction parallel to the line where the two MR sensors have been disposed, as shown in FIG. 37.

More specifically, the probe 102C has a configuration wherein minute vibration of the sensor unit 120 is performed in the direction orthogonal to the longitudinal direction thereof by the actuator 123 driving a joint 124C and a cam 181, formed of non-magnetic material, for example. Note that reference numeral 182 denotes a driver for driving the actuator 123.

With the probe 102C having such a configuration, the MR sensors 122 (sensors MR1 and MR2) are positioned asymmetrically with respect to the magnetic fluid 146 as shown in FIG. 38A, and accordingly, the greatest signals are output from the MR sensors 122 (sensors MR1 and MR2) as shown in FIG. 38B. Note that FIG. 38A is a schematic diagram which illustrates minute vibration of the sensor unit being performed in the direction parallel to the line where the two MR sensors have been disposed, and FIG. 38B is a chart which shows signals from the MR sensors of the sensor unit shown in FIG. 38A.

The signals from the MR sensors 122 (sensors MR1 and MR2) of the probe 102C subjected to demodulation and the Fourier transformation each represent the distortion of the local magnetic field (the change in the magnetic flux density) in the direction parallel to the MR sensors 122 (sensors MR1 and MR2), and the difference between the output signals is obtained, and accordingly, the sensor unit outputs signals from the MR sensors 122 (sensors MR1 and MR2) with the frequency twice as high as the vibration frequency of the sensor unit 120. Note that FIG. 39 is a chart which shows signals from the sensor unit shown in FIG. 38A, which have been subjected to Fourier transformation.

On the other hand, in the event that minute vibration of the sensor unit 120 is performed in the direction orthogonal to the line where the two MR sensors have been disposed, and in the longitudinal direction thereof including the magnet, minute vibration of the MR sensors 122 (sensors MR1 and MR2) is performed only in the longitudinal direction thereof, and the MR sensors 122 are not moved in the direction parallel thereto with the positions thereof maintained symmetrical to the magnetic fluid 146 as shown in FIG. 40A, leading to a problem that the signals from the MR sensors 122 (sensors MR1 and MR2) are dependent upon the vibration frequency as shown in FIG. 40B. Note that FIG. 40A is a schematic diagram which illustrates minute vibration of the sensor unit being performed in the direction orthogonal to the line where the two MR sensors have been disposed, and in the longitudinal direction thereof including the magnet, and FIG. 40B is a chart which shows signals from the MR sensors of the sensor unit shown in FIG. 40A.

In this case, the signals output from the MR sensors 122 (sensors MR1 and MR2) are drowned by noise around the vibration frequency due to contact resistance or the like of the wiring or the like within the sensor unit 120, leading to difficulties in detecting the signals.

However, with the probe 102C according to the sixth embodiment, minute vibration of the sensor unit 120 is performed in the direction orthogonal to the longitudinal direction thereof, and accordingly, minute vibration of the MR sensors 122 (sensors MR1 and MR2) is performed in the direction parallel thereto. Thus, with the probe 102C according to the sixth embodiment, the MR sensors 122 (sensors MR1 and MR2) are positioned asymmetrically with respect to the magnetic fluid 146 as compared with an arrangement wherein minute vibration of the sensor unit 120 is performed in the longitudinal direction thereof, and accordingly, the signals from the MR sensors 122 (sensors MR1 and MR2) are output with a frequency twice as high as the vibration frequency of the sensor unit 120 as described above. Thus, with the probe 102C according to the sixth embodiment, the detected signals are not dependent upon the vibration frequency, thereby detecting clear signals without influence of noise around the vibration frequency.

As a result, the magnetic fluid detecting apparatus according to the present sixth embodiment can detect the magnetic fluid 146 more accurately and efficiently identify the sentinel lymph node 145, as well as having the same advantages as with the above-described fourth and fifth embodiments.

Having described the preferred embodiments of the invention referring to the accompanying drawing, it should be understood that the present invention is not limited to those precise embodiments and various changes and modification thereof could be made by one skilled in the art without departing from the spirit or scope of the invention as defined in the appended claims.

What is claimed is:

1. A magnetic fluid detecting apparatus for identifying sentinel lymph nodes comprising:
a single or a plurality of magnets for exciting magnetic fluid accumulated within the subject; and
a plurality of magnetic sensors for detecting the distortion of the local magnetic distribution due to the magnetic fluid excited by the magnets;
wherein any of the magnet, the combination of the magnet and the plurality of the magnetic sensors, and the combination of the magnet, the plurality of the magnetic sensors, and a preamplifier for amplifying the outputs from the plurality of the magnetic sensors, is vibrated or rotated, and the difference between the outputs from the plurality of the magnetic sensors is obtained and is subjected to demodulation, thereby detecting the magnetic fluid.

2. A magnetic fluid detecting apparatus for identifying sentinel lymph nodes comprising:
a single or a plurality of electromagnets for exciting magnetic fluid accumulated within the subject; and
a plurality of magnetic sensors for detecting the distortion of the local magnetic distribution due to the magnetic fluid excited by the electromagnet;
wherein the electromagnet is driven by AC current, the difference between the outputs from the plurality of the magnetic sensors is obtained, and is subjected to demodulation, thereby detecting the magnetic fluid.

3. A magnetic fluid detecting apparatus for identifying sentinel lymph nodes according to claim 1, wherein the two magnets are disposed in parallel in the same direction of polarity;
and wherein the plurality of magnetic sensors are disposed between the two magnets.

4. A magnetic fluid detecting apparatus for identifying sentinel lymph nodes according to claim 1, wherein the magnet comprises a U-shaped magnet or a horseshoe-shaped magnet, and wherein the plurality of magnetic sensors are disposed so that the U-shaped magnet or the horseshoe-shaped magnet surrounds the plurality of the magnetic sensors.

5. A magnetic fluid detecting apparatus for identifying sentinel lymph nodes according to claim 1, further comprising an actuator for vibrating or rotating any of the magnet, the combination of the magnet and the plurality of the magnetic sensors, and the combination of the magnet, the plurality of the magnetic sensors, and the preamplifier.

6. A magnetic fluid detecting apparatus for identifying sentinel lymph nodes according to claim 1, wherein the magnet, the magnetic sensors, the actuator, and the preamplifier, are included in a single watertight probe formed of non-magnetic material.

7. A magnetic fluid detecting apparatus for identifying sentinel lymph nodes according to claim 2, wherein the electromagnet is disposed and driven such that the electromagnet has small influence of the magnetic field upon the magnetic sensors, and has great influence of the magnetic field upon the magnetic fluid, due to the interaction of the magnetic field formed by the electromagnet.

8. A magnetic fluid detecting apparatus for identifying sentinel lymph nodes according to claim 2, wherein the electromagnet has a ferrite magnetic core formed of a U-shaped magnet or a horseshoe-shaped magnet, and wherein the plurality of magnetic sensors are disposed so that the U-shaped magnet or the horseshoe-shaped magnet surrounds the plurality of magnetic sensors.

9. A magnetic fluid detecting apparatus for identifying sentinel lymph nodes according to claim 2, further comprising a driving circuit for driving the electromagnet.

10. A magnetic fluid detecting apparatus for identifying sentinel lymph nodes according to claim 2, wherein the electromagnet, the magnetic sensors, and the preamplifier, are included in a single watertight probe formed of non-magnetic material.

11. A magnetic fluid detecting apparatus for identifying sentinel lymph nodes according to claim 2, wherein the magnetic sensors are formed of thin-film sensors, and the electromagnet is formed of a thin-film coil and a thin-film magnetic material; and wherein these components are formed on the same semiconductor substrate as the preamplifier.

12. A magnetic fluid detecting apparatus for identifying sentinel lymph nodes according to claim 7, wherein the plurality of electromagnets are disposed at the positions where the change of the magnetic field over time does not occur, and wherein magnetic sensors are disposed at the positions where the change of the magnetic field does not occur.

13. A magnetic fluid detecting apparatus for identifying sentinel lymph nodes according to claim 7, wherein the electromagnet is formed of two electromagnets, one great and one small, disposed concentrically with each other.

14. A magnetic fluid detecting apparatus for identifying sentinel lymph nodes according to claim 7, wherein the electromagnet is formed of two electromagnets disposed in parallel, with current applied to the electromagnets so that the polarities are formed in the same direction, and wherein the magnetic sensors are disposed between the electromagnets and oriented so as not to be influenced by the magnetic field therefrom.

15. A magnetic fluid detecting apparatus for identifying sentinel lymph nodes according to claim 4, wherein the magnetic sensor comprises an MI (Magnetic Impedance) sensor or an MR (Magnetic Resistance) sensor.

16. A magnetic fluid detecting apparatus for identifying sentinel lymph nodes according to claim 8, wherein the magnetic sensor comprises an MI (Magnetic Impedance) sensor or an MR (Magnetic Resistance) sensor.

17. A magnetic fluid detecting apparatus for identifying sentinel lymph nodes according to claim 5, further comprising a driver for driving the actuator, wherein the driver or the preamplifier is magnetically shielded.

18. A magnetic fluid detecting apparatus for identifying sentinel lymph nodes according to claim 9, wherein the driving circuit is magnetically shielded.

19. A magnetic fluid detecting apparatus for identifying sentinel lymph nodes according to claim 10, wherein the preamplifier is magnetically shielded.

20. A magnetic fluid detecting apparatus for identifying sentinel lymph nodes according to claim 12, wherein the plurality of electromagnets are disposed symmetrically in the vertical direction, with a single or a plurality of magnetic sensors for the electromagnets being disposed for detecting the magnetic field formed by the plurality of the electromagnets;
and wherein feedback is performed for a predetermined electromagnet of the plurality of electromagnets so that the outputs from the magnetic sensors for the electromagnets or the sum of these outputs are maintained constant.

21. A magnetic fluid detecting apparatus for identifying sentinel lymph nodes comprising:
a single or a plurality of magnets for exciting magnetic fluid accumulated within the subject;
a plurality of magnetic sensors for detecting the distortion of the local magnetic distribution due to the magnetic fluid excited by the magnets; and
a variable offset unit for offsetting the difference between the outputs from the plurality of the magnetic sensors;
wherein any of the magnet, the combination of the magnet and the plurality of the magnetic sensors, and the combination of the magnet, the plurality of the magnetic sensors, and a preamplifier for amplifying the outputs from the plurality of the magnetic sensors, is vibrated or rotated, and the difference between the outputs from the plurality of the magnetic sensors is obtained and is subjected to demodulation, thereby detecting the magnetic fluid;
and wherein the variable offset unit adjusts the amount of the offset based upon the output from the preamplifier.

22. A magnetic fluid detecting apparatus for identifying sentinel lymph nodes according to claim 21, wherein the variable offset unit comprises a CdS photocoupler.

23. A magnetic fluid detecting apparatus for identifying sentinel lymph nodes comprising:
a single or a plurality of magnets for exciting magnetic fluid accumulated within the subject;
a plurality of magnetic sensors for detecting the distortion of the local magnetic distribution due to the magnetic fluid excited by the magnets; and
a variable offset unit for offsetting the difference between the outputs from the plurality of the magnetic sensors;
wherein any of the magnet, the combination of the magnet and the plurality of the magnetic sensors, and the combination of the magnet, the plurality of the magnetic sensors, and a preamplifier for amplifying the outputs from the plurality of the magnetic sensors, is vibrated or rotated, and the difference between the outputs from the plurality of the magnetic sensors is obtained and is subjected to demodulation, thereby detecting the magnetic fluid;
and wherein the plurality of the magnetic sensors and the preamplifier form an AC coupling configuration.

24. A magnetic fluid detecting apparatus for identifying sentinel lymph nodes comprising:
a single or a plurality of magnets for exciting magnetic fluid accumulated within the subject; and
a plurality of magnetic sensors for detecting the distortion of the local magnetic distribution due to the magnetic fluid excited by the magnets;
wherein any of the magnet, the combination of the magnet and the plurality of the magnetic sensors, and the combination of the magnet, the plurality of the magnetic sensors, and a preamplifier for amplifying the outputs from the plurality of the magnetic sensors, is vibrated in the direction parallel to a line or a plane including the plurality of the magnetic sensors, and the difference between the outputs from the plurality of the magnetic sensors is obtained and is subjected to demodulation.

25. A magnetic fluid detecting apparatus for identifying sentinel lymph nodes according to claim 24, further comprising a variable offset unit for offsetting the difference between the outputs from the plurality of the magnetic sensors, wherein the variable offset unit adjusts the amount of the offset based upon the output from the preamplifier.

26. A magnetic fluid detecting apparatus for identifying sentinel lymph nodes according to claim 24, wherein the plurality of the magnetic sensors and the preamplifier form an AC coupling configuration.

27. A magnetic fluid detecting apparatus for identifying sentinel lymph nodes according to claim 25, wherein the variable offset unit comprises a CdS photocoupler.
